(19) United States
(12) Patent Application Publication
(10) Pub. No.: US 2026/0249033 A1
(43) Pub. Date: Aug. 27, 2026

(54) A POSITIVE AIRWAY PRESSURE DEVICE

(71) Applicant: TASMANIAN MEDICAL INNOVATIONS PTY LTD, Tasmania (AU)

(72) Inventors: Kathleen KAI LING LIM, Tasmania (AU); Peter Anderson DARGAVILLE, Tasmania (AU); Lachlann MCLEOD, Tasmania (AU); Andrew Patrick MARSHALL, Tasmania (AU); Cameron Peter Stanley KEATING, Tasmania (AU)

(21) Appl. No.: 18/714,490

(22) PCT Filed: Nov. 29, 2022
(86) PCT No.: PCT/AU2022/051428

§ 371 Date: May 29, 2024

(30) Foreign Application Priority Data
Nov. 29, 2021 (AU) 2021903846

(51) Int. Cl. A61M 16/06; A61M 16/00

(52) U.S. Cl. CPC A61M 16/0605; A61M 16/0051; A61M 16/0694; A61M 2205/3331; A61M 2205/583

(57) ABSTRACT

The invention is broadly directed to a series of sensors for measurement of the pressure of contact of a face mask or other respiratory interface in a positive airway pressure device. Contact pressure measurements are performed through a single or an array of sensors which can be embedded into or coupled onto the positive airway pressure device/system to assess the contact pressure and/or its regional distribution applied to the subject by the respiratory interface. The invention includes the capacity to process the measured information along with measured airway pressure to allow closed-looped adjustment of the mask position and application to the face.

### CROSS-REFERENCE TO RELATED APPLICATIONS

This application is the National Stage filing under 35 U.S.C. 371 of International Application No. PCT/AU2022/051428 filed Nov. 29, 2022, which claims the benefit of earlier filing date and right of priority to Australian Application No. 2021903846 filed on Nov. 29, 2021, the contents of which are all hereby incorporated by reference herein in their entirety.

### FIELD

The disclosure relates to a positive airway pressure device providing continuous monitoring for suboptimal airway pressure delivery and/or suboptimal interface application events. The disclosure also relates to a positive airway pressure device providing an automated adjustment system to correct suboptimal airway pressure and/or interface application events. The disclosure further relates to a positive airway pressure device providing a configurable user interface.

### BACKGROUND

Continuous positive airway pressure (CPAP) is a form of mechanical respiratory support in which a continuous level of pressure above atmospheric pressure is applied to the upper airway of a subject. While used to treat sleep apnoea, CPAP may also be used to treat preterm infants whose respiratory systems have not yet fully developed.

Globally, over 15 million infants are born prematurely every year and are at higher risk of respiratory distress syndrome as the surfactant required to facilitate efficient breathing is not developed until the final weeks of a pregnancy. Without sufficient surfactant, the alveoli in the lungs cannot function properly and are at risk of collapse, resulting in the supply of oxygen to the body becoming compromised. CPAP therapy can assist in lung function until such time as the subject is capable of sustained normal breathing.

Known CPAP devices suffer from a range of failings, in the main related to the difficulty applying an interface (usually a mask) of generic shape to the face of an individual subject and maintaining it in position. If the mask is not mounted properly, it may slide in and out of position during use, resulting in reduced efficiency. If the mask is secured too loosely a seal over the airway may not be maintained, resulting in a failure to deliver sufficient positive pressure to the subject, with the possibility of destabilising effects including loss of oxygenation and even brain injury or death. Conversely, the mask of the device may be secured too tightly on the face, resulting in discomfort and also in pressure injury to the skin and underlying tissue, which can lead to permanent facial scarring. These failings are common to CPAP applied in any subject, but are particularly problematic, both because of their likelihood and the potential for ill-effects, in premature babies ranging from 22-36 weeks' gestation.

The present disclosure was conceived with these shortcomings in mind.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, a limited number of the exemplary methods and materials are described herein.

### SUMMARY

In a first aspect, the disclosure provides a mask for use with a positive airway pressure device, the mask comprising a cap, a frame, and a cushion together forming a chamber configured to be positively pressurised by a source of positively pressurised air, the frame comprising an aperture for communicating the source of positively pressured air into the chamber of the mask adjacent to the nares and/or mouth of a subject, the cushion extending around a periphery of the cap defining a lip and a sealing surface, wherein the lip supports at least one sensor and the sealing surface seals the chamber to the nares and/or mouth, the at least one sensor configured to assess contact pressure applied to the subject's face by the sealing surface of the cushion.

The at least one sensor may be configured to measure force or pressure applied thereto. In some embodiments, the at least one sensor may be configured to vary an output characteristic in response to a measured change in force or pressure applied thereto. For example, in one embodiment the at least one sensor may be a force sensitive resistor, wherein electrical resistance of the sensor decreases in response to increased force applied to the sensor. In some embodiments, the at least one sensor may be a pressure transducer that generates a signal or reading as a function of the pressure applied thereto.

The term “contact pressure” as used herein is understood to define the pressure applied to the surface of a subject's face by the mask in use. Contact pressure may also be measured as a force (the product of the contact pressure and the area of the region over which it is locally applied). Contact pressure may be determined in absolute terms for regions of the face in contact with the mask, and may also be measured in relative terms, wherein the pressure in a given region may be expressed as a proportion of that of a reference point, or in proportion to the overall value. This contact pressure can be applied anywhere on the face of the subject depending on where the mask contacts the subject. In some embodiments, where a nasal mask is used, the contact pressure will be experienced around the nares of the subject. Where a mask covers the nares and mouth of a subject, the contact pressure will be experienced around the nasal area and mouth of a subject. Where a full-face mask is used the contact pressure will typically be experienced around the cheeks, forehead and mouth and jaw of the subject.

In some embodiments the array of sensors may be arranged to measure the contact pressure indirectly. The sensors may be configured to transduce the deformation, strain, stretching, or bending of components of the mask assembly, including the mask, frame, or straps, from which the contact pressure for regions of the mask can be measured, derived or inferred. In some embodiments a plurality of sensors may be mounted to the internal or external surfaces of the frame of the mask. Alternatively, a plurality of sensors may be mounted to or embedded in the straps.

The mask may be configured to work in conjunction with a monitor comprising a processor that receives and processes readings from the at least one sensor. The processor may receive or calculate from the readings the contact pressure applied to the subject from the at least one sensor and subsequently compare the contact pressure against a predetermined contact pressure range to categorise the at least one sensor as being: within the predetermined contact pressure range, below the predetermined contact pressure range or above the predetermined contact pressure range, and displaying the category on the monitor. This provides a medical practitioner real time information as to whether the mask is applying the correct contact pressure, too much contact pressure or insufficient contact pressure to the subject to deliver optimal airway support. The predetermined contact pressure range is dependent on the age, size, and condition of the subject. Similarly, relative contact pressure in each of the sensors may be compared with a desired range, as may the proportional distribution of contact pressures.

While the mask as described herein refers to the delivery of positively pressurised air, it is understood that the air delivered can be air or an oxygen enriched air. The oxygen level can be increased from 21% to 100% as required by the subject.

In some embodiments a plurality of sensors is disposed around the mask lip forming a sensor array to enable more detailed monitoring of the contact pressure applied to the subject and identify and quantify the regional distribution of contact pressure. In addition, some embodiments of the disclosure utilise algorithmic control for linking to systems for automated correction of regional absolute or relative contact pressure and/or the proportional distribution of contact pressure, both for overcoming airway pressure loss and for the correction of unacceptably high regional contact pressure. The “airway pressure” or “delivered airway pressure” is a measure of the pressure within the air circuit connected to the mask.

The array of sensors may be mounted on an external surface of the mask lip, externally of the chamber. Alternatively, the array of sensors may be mounted on an internal surface of the mask lip, internally of the chamber. Alternatively, the array of sensors may be embedded into the material of the mask lip. In some embodiments, the array of sensors may be embedded within a sleeve or a conformal layer that is configured to shroud the cushion of the mask and/or the subject.

The sealing surface may be configured to continuously surround the nares of the subject and form a seal with the subject's face. In some embodiments the sealing surface may be configured to surround the mouth and nares of the subject and form a seal with the subject's face. In some embodiments, the sealing surface may surround the mouth, nares and eyes of the subject forming a full-face mask.

Each of the plurality of sensors may be mounted adjacent to an LED, configured to illuminate when the adjacent sensor measures a contact pressure between the subject and the sealing surface of the mask outside of the predetermined contact pressure range.

The acceptable absolute or relative contact pressure range and the acceptable range for proportional distribution of contact pressures around the mask may be set by a medical practitioner for a given subject, and are bounded by an accepted maximum value and an accepted minimum value.

The adjacent LED may be configured to emit red light when a measure of the contact pressure between the subject and the sealing surface of the mask rises above the acceptable contact pressure range. The measure of the contact pressure may be absolute or relative. The adjacent LED may be configured to emit blue light when the contact pressure reading between the subject and the sealing surface of the mask falls below the acceptable contact pressure range. The adjacent LED may be configured to emit green light when the contact pressure reading between the subject and the sealing surface of the mask is within the acceptable contact pressure range.

Each of the plurality of sensors may be wired into an electrical harness transmitting sensor data to the processor, wherein the processor receives the measure of the contact pressure from the sensor data for each sensor and determines output data for each sensor in one of three pressure states: above the acceptable contact pressure range; below the acceptable contact pressure range; and within the acceptable contact pressure range.

The harness may terminate with a connector configured to cooperatively engage with at least one of the processor, a pre-processor, a data port, or the monitor. The force readings from the sensors may be transmitted to the pre-processor for filtering of the sensor data before being sent to the processor for processing. Output data from the processor may be directed to the monitor, which is configured to display the category for each sensor. In some embodiments, the output data is wirelessly transmitted to the monitor using, for example, radio waves, Bluetooth, Wi-Fi, NFC, LAN, Wan, and other forms of mobile network. In some embodiments, the wireless transmission of the output data may also be sent directly to a portable electronic device, such as a computer, a laptop, a smart phone, a pager, a tablet etc.

The output data categorises the value arising from each sensor as being: within the predetermined contact pressure range, above the predetermined contact pressure range, and below the predetermined contact pressure range. The pressure state for each contact pressure reading may be colour-coded on the monitor or directed to a graphical user interface. The acceptable (absolute or relative) contact pressure range may be input to the processor via an input device associated with the monitor (e.g. a touch screen, a keyboard, customised buttons or a microphone). In some embodiments the acceptable contact pressure range may be calculated from clinical data supplied via the user interface by a medical practitioner, e.g. age, gestation, and weight. An acceptable contact pressure range for proportional distribution of contact pressures around the mask may be input to the processor. In some embodiments the range of acceptable contact pressure distribution may be calculated from clinical data supplied via the user interface by a medical practitioner (e.g. age, gestation, and weight).

The monitor may provide a quantitative display providing repeatedly updated contact pressure readings for each of the plurality of sensors. In some embodiments the display may be qualitative, providing a traffic-light colour coding, for example red indicating an excessive contact pressure, green indicating contact pressure within range, and blue indicating a suboptimal contact pressure. The monitor may provide a feedback signal in response to a sensor reading from any one of the sensors outside of the acceptable contact pressure range. The feedback signal may be at least one of a visual alert, an audible alert, a haptic alert, a text message or any combination thereof.

In one embodiment, the cushion may comprise a plurality of fluid-filled chambers or compartments, each compartment individually connected with a supply of gas or liquid to increase or decrease the volume of each compartment independently of each of the remaining compartments. Fluid may be supplied to or drawn from selected fluid-filled compartments in response to the pressure state for each sensor determined by the processor. The fluid may be supplied to at least one fluid-filled compartment adjacent a first sensor in response to the processor categorising the contact pressure at the first sensor location as being below the predetermined contact pressure range. The fluid may be drawn from at least one fluid-filled compartment adjacent a first sensor in response to the processor categorising the contact pressure at the first sensor location as being above the predetermined contact pressure range. A fluid volume may be maintained in at least one fluid-filled compartment adjacent a first sensor in response to the processor categorising the contact pressure at the first sensor location as being within the acceptable (absolute or relative) contact pressure range.

A pump may be configured to be actuated in response to output data from the processor to either introduce or remove fluid from selected compartments of the plurality of fluid-filled compartments to thereby increase or reduce contact pressure applied to the subject by the sealing surface of the mask.

Keeping the contact pressure in the acceptable contact pressure range does two things:
(i) Avoids or ameliorates pressure injury related to over-tight mask application.
(ii) Avoids or ameliorates loss of airway pressure, related to the mask being applied too loosely.

The acceptable range for contact pressures and/or proportional distribution is compared with feedback from both the contact pressure sensors, and the airway pressure sensor. Avoiding pressure related injuries and loss of airway pressure is important to all the embodiments of the mask and device as described herein using active monitoring or feedback controls to adjust the mask contact pressure and/or pressure distribution whether that is using the compartmentalised cushion of mask, using adjustable straps, or employing fluid bladders alongside the straps.

When fitting the mask to the subject for the first time, the individual fluid-filled compartments may be adjusted to better conform to the contours of the subject's face before setting the desired contact pressure range for monitoring, thereby providing a configurable sealing surface.

In some embodiments, the frame of the mask may comprise a tether or tethers for connecting one or more straps to the mask.

In a second aspect, the disclosure provides an adjustable mask assembly, including a mask as described herein and adjustable straps for applying the chamber of the mask to the face adjacent to the nares and/or mouth of the subject, the adjustable straps comprising: a cavity for receiving and retaining a fluid therein, wherein introduction of fluid into the cavity increases the local contact pressure applied to the subject by the sealing surface of the mask.

In some embodiments, the mask may be configured to surround the subject's nares only. In alternative embodiments, the mask may be configured to surround the nares and mouth of the subject. In still further embodiment, the mask may be configured to surround the subject's face covering the nares, mouth and eyes.

In response to the contact pressure at each sensor location around the cushion, whether directly measured or calculated from the sensor data, and in combination with the measured airway pressure, the processor may be configured to initiate a pump to augment or decrease the fluid volume within the cavity of the adjustable straps to thereby vary the contact pressure applied to the subject by the mask. In this manner, the disclosure provides automated adjustment to the adjustable straps to maintain the predetermined contact pressure or contact pressure distribution around the subject's face.

The pump may be actuated in response to the output data from the processor to either introduce or remove fluid from the cavity of the adjustable straps to thereby increase or reduce contact pressure applied to the subject by the sealing surface of the mask.

Extraction of fluid from the cavity may decrease the contact pressure applied to the subject by the sealing surface of the mask. Conversely, pumping fluid into the cavity may increase the contact pressure applied to the subject by the sealing surface of the mask. The fluid may be a liquid or a gas.

In some embodiments, the cavity of the strap may include an impermeable bladder for receiving and retaining the fluid. The bladder may be embedded or attached to the inner or outer surface of the strap, and may be retrofitted to existing straps.

The fluid may be sourced from a reservoir via the pump configured to introduce or extract fluid from the cavity in response to the output signal from the processor. The fluid may be provided in a canister, or air canister, to provide a dedicated air supply to the cavity.

The adjustable strap may be configured to pass around the head of the subject. The adjustable strap may be configured to operate in a first plane drawing the mask towards the subject. The adjustable strap may comprise a secondary limb, the secondary limb configured to operate in a second plane. The secondary limb may be engageable with the adjustable strap to tension the mask toward the subject in the second plane. The secondary limb may comprise a secondary cavity for receiving and retaining a fluid therein to adjust contact pressure applied to the subject by the sealing surface of the mask.

In a third aspect, the disclosure provides a positive airway pressure device, for monitoring contact pressure applied to a subject, the device comprising a mask supported on an armature the mask having a chamber for directing a positive pressure air supply to the nares and/or mouth of the subject, and providing a plurality of sensors configured to assess contact pressure applied to the subject by the mask, the armature comprising: a respiratory channel and an expiratory channel for supplying positive pressure air supply to the chamber and evacuating expelled air therefrom; a port for receiving sensor data from the plurality of sensors and directing the sensor data to a processor, wherein the processor compares the sensor data from each sensor against a predetermined contact pressure range and determines output data for each sensor indicating whether the contact pressure falls outside of a predetermined contact pressure range.

The contact pressure may be measured or calculated in different ways, including in absolute or relative terms. The regional distribution of the contact pressure applied to a subject may also be measured.

The processor uses a proprietary algorithm to enable identification and notification of a loss of airway pressure. The term “airway pressure” as used herein is understood to define the pressure of the air delivered to the mask and particularly within the chamber of the mask by the CPAP device, which is then delivered to the upper airway of the subject. The term “loss of airway pressure” as used herein is understood to include any drop or reduction in the pressure of the air supplied to the mask that renders a decrease in airway pressure outside of a predetermined pressure range for a given subject and for a set level of CPAP.

The output data from the processor may be directed to a monitor configured to indicate where the contact pressure for any one of the plurality of sensors falls outside of the predetermined contact pressure range and whether the airway pressure is remaining constant or dropping. The processor may be housed within the monitor. The sensor data may be sent to a first pre-processor to be filtered before being transmitted to the processor.

The monitor may have a qualitative display repeatedly providing a visual indication of the categorisation of the contact pressures for each of the plurality of sensors, and the proportional distribution of these pressures around the sensor array. In some embodiments, the monitor may have a quantitative display repeatedly providing updated measures of contact pressures and/or proportional distribution for each of the plurality of sensors. The contact pressure may be measured or calculated in different ways, including in absolute or relative terms. The regional distribution of the contact pressure applied to a subject may also be measured. The monitor may also indicate the airway pressure as delivered to the mask. The monitor may display a quantitative value for the airway pressure. In some embodiments the airway pressure may be set, defining an acceptable minimum level and an acceptable maximum level for the subject, such that the monitor indicates when the airway pressure changes or moves outside of the range defined by the acceptable minimum and maximum levels.

The monitor may provide a feedback signal in response to contact pressures at any one of the plurality of sensors falling outside of the predetermined contact pressure range.

The plurality of sensors may be arranged peripherally about the cushion of a mask to form an array. The array of sensors configured to assess contact pressure applied to the subject by the mask at a plurality of locations proximal to the nares and/or mouth of the subject. The array of sensors may be visually represented on the monitor both spatially and/or colour-coded to indicate the contact pressure in absolute or relative terms for each sensor in the array, categorised in relation to acceptable ranges.

The array of sensors may comprise only one or two sensors, depending on the size of the mask. In some embodiments the sealing surface of the mask may be covered by 3 sensors. In some embodiments, the sealing surface of the mask may comprise 6 sensors. In some embodiments, the sealing surface may support 10 or more sensors.

An LED may be arranged adjacent each of the plurality of sensors and configured to emit a coloured light in response to the output data from the processor.

The armature may further comprise an access port for air sampling from at least one of the respiratory channel and the expiratory channel. The access port may be configured for medication delivery. The access port may be sealable.

In some embodiments, the armature may further comprise a filter. In some embodiments the armature may further comprise an airway pressure sensor, to monitor the pressure of the positively pressurised air within the chamber of the mask.

Each of the plurality of sensors may be wired into a harness comprising a plurality of electrical cables, each cable connected with one of the plurality of sensors. The harness may provide a lead terminating with a connector configured to cooperatively engage with a data port of the armature. The connector facilitates disconnection of the mask and harness from the device for repair, replacement and cleaning. The data port may transfer the sensor data from the lead to a data cable. The data cable extends the length of the device and transmits the sensor data to the processor. The output data from the processor may then be transferred to the monitor via an output cable.

In some embodiments, the output data from the processor may be wirelessly transmitted to the monitor using, for example, radio waves, Bluetooth, Wi-Fi, NFC, LAN, WAN, and other forms of mobile network. In some embodiments, the wireless transmission of the out data may also be sent directly to a portable electronic device, such as a computer, a laptop, a smart phone, a pager, a tablet etc.

In some embodiments, the device may further comprise a strap for securing the lead thereto. The device may further comprise at least one supplementary strap for constraining at least one of the lead, the data cable and the output cable to the armature.

In a fourth aspect, the disclosure provides a method of monitoring the delivery of positive airway pressure via a mask to a subject, the method comprising the steps of: placing a mask over the nares and/or mouth of the subject, the mask comprising at least one sensor configured to assess contact pressure applied to the subject by the mask; transmitting data from the at least one sensor to a processor for calculating the contact pressure applied to the subject by the mask; defining a predetermined contact pressure range via a monitor, to facilitate characterisation of the at least one sensor into any one of three categories: contact pressure below the predetermined contact pressure range, contact pressure above the predetermined range, and contact pressure within the predetermined contact pressure range; initiating the supply of positively pressurised air to the mask; and selecting an operational mode of the monitor determining actions to be taken in response to the categorisation of the at least one sensor.

The contact pressure may be obtained or measured/calculated in absolute or relative terms. In some embodiments, the at least one sensor may be configured to obtain a regional distribution of the contact pressure, or an indirect measurement of contact pressure and its distribution using stretch or flex sensors, or strain gauges.

In some embodiments, the method further comprises the step of monitoring the airway pressure of the positively pressurised air delivered to the mask. The method may further include the step of triggering an alarm when the airway pressure drops below a predetermined allowable minimum pressure for the subject. The method may further include the step of triggering an alarm when the airway pressure increases above a predetermined maximum allowable pressure for the subject. The predetermined minimum allowable airway pressure and predetermined maximum allowable airway pressure may be adjusted for a given subject.

In each of the above aspects and embodiments, the device may include additional sensors to monitor other characteristics associated with the mask, such as the airway pressure.

If the sensed airway pressure is outside acceptable bounds, this will usually indicate an error in the fitting of the mask, and may indicate that air is escaping out of the mask. In that event, the contact pressure (absolute or relative) measured at the site of each sensor can be checked, to determine the location where air is escaping. This information can be displayed to a user—to indicate that there is an issue or problem with the airway pressure within the mask, and/or the location of the site of the problem. In some embodiments, the present disclosure may automatically adjust the positioning of the mask, for example by adjusting adjustable straps, to correct any problem that is identified-such as air escaping due to insufficient contact at a particular sensor site. The present disclosure can thereby facilitate the correction of a detected problem.

Various features, aspects, and advantages of the disclosure will become more apparent from the following description of embodiments of the disclosure, along with the accompanying drawings in which like numerals represent like components.

### BRIEF DESCRIPTION OF THE DRAWINGS

Embodiments of the disclosure are illustrated by way of example, and not by way of limitation, with reference to the accompanying drawings, of which:

FIG. 1 is a perspective view of a mask according to one embodiment of the disclosure, illustrating a harness for transmitting data from a plurality of sensors;

FIG. 2 is a perspective view of the mask of FIG. 1, illustrating the plurality of sensors equidistantly spaced about a cushion of the mask;

FIG. 3 is an underside view of the mask of FIG. 1, illustrating the peripheral cushion or lip forming the contact surface of the mask on the face of the subject, and showing the position of an array of six contact pressure sensors;

FIG. 4A is a plan view of a six sensor harness connecting the sensor array shown in FIG. 3, prior to attachment to a mask;

FIG. 4B is a perspective view of a mask (in shadow outline) illustrating the six sensor harness mounted to the frame of the mask;

FIG. 4C is a perspective view of the mask indicating an opening for a lead of the harness to exit the frame of the mask;

FIG. 5A is a side view of a mask according to one embodiment engaged with an armature delivering air to the chamber of the mask which when applied to the face delivers positive pressure to the airway;

FIG. 5B is a top view of the mask of FIG. 5A having a plurality of stretch sensors across a cap of the mask, enabling indirect measurement of contact pressure;

FIG. 5C is a bottom view of the mask of FIG. 5A illustrating a cushion surrounding the mask to, in use, form a sealing surface with a subject;

FIG. 6A is a side view of a mask according to one embodiment, engaged with the armature, the mask providing a plurality of flex sensors disposed around the cushion, enabling the indirect measurement of contact pressure;

FIG. 6B is a bottom view of the mask of FIG. 6A illustrating the plurality of sensors, disposed around the cushion;

FIG. 7A is a side view of a mask according to one embodiment, engaged with the armature, the mask providing strain gauge sensors around the frame of the mask;

FIG. 7B is a schematic view of a cross-section through the frame of the mask of FIG. 7A, indicating the location of the strain gauges measuring strain on an external surface of the mask;

FIG. 7C is a schematic view of a cross-section through the frame of the mask of FIG. 7A, indicating the location of the strain gauges measuring strain on external and internal surfaces of the mask;

FIG. 8A is a mask according to another embodiment of the disclosure the mask having a peripheral cushion comprising a plurality of fluid-filled compartments, each compartment having an independent fluid supply;

FIG. 8B is an underside view of the mask of FIG. 8A, illustrating the plurality of fluid-filled chambers in plan view;

FIG. 9 is a perspective view of a continuous positive airway pressure device according to an embodiment of the disclosure, comprising a mask supported on an armature;

FIG. 10 is a perspective view of the device of FIG. 9 operatively connected to the airway of a subject, illustrating an adjustable head mounting;

FIG. 11 is a schematic view of the adjustable strap of FIG. 10 for securing the mask to the subject, illustrating a fluid cavity within the adjustable strap for receiving fluid therein;

FIG. 12A is a side view of the device indicating optional mounting sites on the mask and armature for engagement with the adjustable strap;

FIG. 12B is a plan view of a strap according to one embodiments, illustrating a stretch or strain sensor extending along the strap;

FIG. 13A is an adjustable strap according to one embodiment, illustrating a plurality of fluid compartments evenly expanded along a length of the strap;

FIG. 13B is the adjustable strap of FIG. 13A, illustrating volume reduction in three of the fluid compartments to reduce the length of the strap;

FIG. 14 is a monitor for the device, including a visual representation of the sensor array around the contact surface of the mask, each sensor in the array colour-coded to indicate pressure state of the sensor; and

FIG. 15 is a flow chart setting out a method of monitoring a supply of positively pressurised air to a subject; and

FIG. 16 is a schematic layout of the device according to one embodiment of the disclosure.

Embodiments will now be described more fully hereinafter with reference to the accompanying drawings, in which various embodiments, although not the only possible embodiments, of the disclosure are shown. The disclosure may be embodied in many different forms and should not be construed as being limited to the embodiments described below.

### DETAILED DESCRIPTION

Whilst the disclosure is described herein in relation to a mask for use with a positive airway pressure device, it is contemplated that the mask can be used with other medical devices and breathing apparatus or used in isolation, to thereby monitor forces applied to a subject while the mask is in use, with application to masks used for respiratory support, underwater diving and for oxygen delivery during firefighting and other emergency situations.

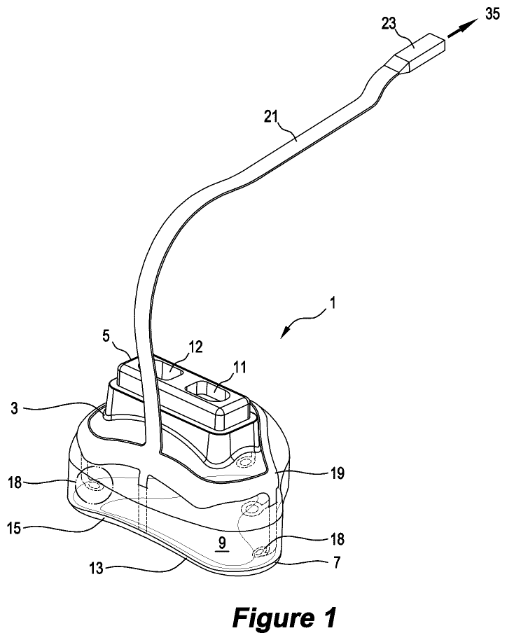

With reference to FIGS. 1-3, there is illustrated a mask (1) for use with a positive airway pressure device (100), the mask (1) comprising a cap (3), a frame (5), and a cushion (7) together forming a chamber (9) configured to be positively pressurised by a source of pressurised air, the frame (5) including an aperture (11) for communicating the source of pressurised air into the chamber (9) adjacent to the nares and/or mouth of a subject, the cushion (7) extending around the periphery of the cap (3), the cushion having a lip (15) supporting a plurality (17) of sensors (18) and a sealing surface (13) sealing the chamber (9) against the face of the subject, wherein each of the sensors (18) is configured to assess contact pressure applied to the subject by the sealing surface (13) of the cushion (7).

The mask (1) of FIG. 1 is configured to surround the nares of the subject to maintain positive airway pressure within the chamber (9). However, it is contemplated that in some embodiments both the nares and mouth can be covered, and in some embodiments the entire face of the subject can be covered by the mask (1).

The mask (1) comprises the frame (5) which defines at least one aperture for receiving pressurised air. In FIG. 1, the frame (5) includes a pair of apertures (11, 12). A first of the apertures (11, 12) defining an air inlet from the inspiratory limb and a second of the apertures (11, 12) defining an air outlet to the expiratory limb. The air inlet and air outlet are not fixed in relation to the apertures (11, 12) and may vary to match the circuit flow requirements of the subject.

The frame (5) of the mask (1) can be made separately or can be integrally formed with the cap (3) of the mask (1). The cap (3) is domed to define the chamber (9) therein. Illustrated in FIGS. 1-3 the cap (3) is tri-lobed having a first centrally disposed apex for accommodating a bridge of the nose of the subject, and two opposing lobes for covering the nares. In larger masks intended to cover the nares and mouth, the first centrally disposed apex can accommodate a nose of the subject, and the two opposing lobes will loosely mirror the mouth of the subject.

About the periphery of the mask (1) is the cushion (7) which provides the interface to be applied to the face of the subject. The cushion (7) in FIGS. 1-3 has a lip (15) that curls internally (3) turning through about 90 degrees to form the external sealing surface (13) with the subject. All contact with the face of the subject is made via the sealing surface (13). Internally of the chamber (9) the lip (15) supports the plurality of sensors (17) arranged equidistantly about the lip (15).

Each sensor (18) is configured to assess the contact pressure applied to the subject by the sealing surface (13) of the mask. High contact pressure can damage the skin around the nares, and also cause damage to the tissue under the skin resulting in permanent scarring. The contact pressure can be measured or calculated continuously or at repeated intervals, depending on the medical practitioner's direction.

The sensors (18) can measure load/force data and feed the sensor data into a wiring harness (19) that is mounted to the mask (1), to generate continuous force readings. From the force readings, and the known sensor active area, the contact pressure can be calculated. The sensors (18) can also be selected to directly monitor pressure, or to monitor a characteristic of the sensor that varies in response to pressure, such as electrical resistance.

The sensors (18) can be arranged in an array and attributed to regions or zones of the subject's face. The information received from each sensor (18) where attributed to a region will then be used to determine the focus to which any action needs to be taken, whether that action is manual or automated.

The harness (19) is disposed around the cap (3) and on a first side provides a plurality of electrical tendrils (20) extending around the lip (15) to connect with each individual sensor (18). On a second opposing side of the harness (19) an electrical conduit or lead (21) transmits the sensor data from the harness (19).

The sensors (18) can be piezoelectric, capacitive, or resistive sensors and can measure force applied to the subject whether placed in direct contact with the subject or through the lip (15) of the mask (10). It is contemplated that the sensors (18) can be pressure sensitive membrane switches or simple force sensitive resistors. Force sensitive resistors react to an increase in force applied to an active surface area by exhibiting a decrease in resistance.

In one form, the sensors (18) are single axis force sensors providing between 2-15 mm² in active surface area and typically measure changes in force in a single axis, i.e. between the sealing surface of the mask and the subject's face. Each sensor comprises a pair of finger electrodes for transmitting electronic data to a processor (35) and a sensor head for taking a force reading from which contact pressure can be determined in relation to a localised region of the face adjacent to the sensor.

In another form, the sensors (18) are configured to measure the resistance of a force sensitive material disposed between a pair of electrodes, which may be interdigitated fingers, plates or alternative interleaved configuration.

The reading or data from each sensor (18) is transmitted to the processor (35) for filtering, algorithm input and calculations to categorise each sensor (18) and provide clinically relevant information to a monitor (57). In some embodiments the sensor data is transmitted to a pre-processor (36) before being transmitted to the processor (35), the processor (35) being housed within the monitor (57). The processing of the data from the sensors can be filtered in the pre-processor (36) before being transmitted to the processor (35) for calculation and manipulation.

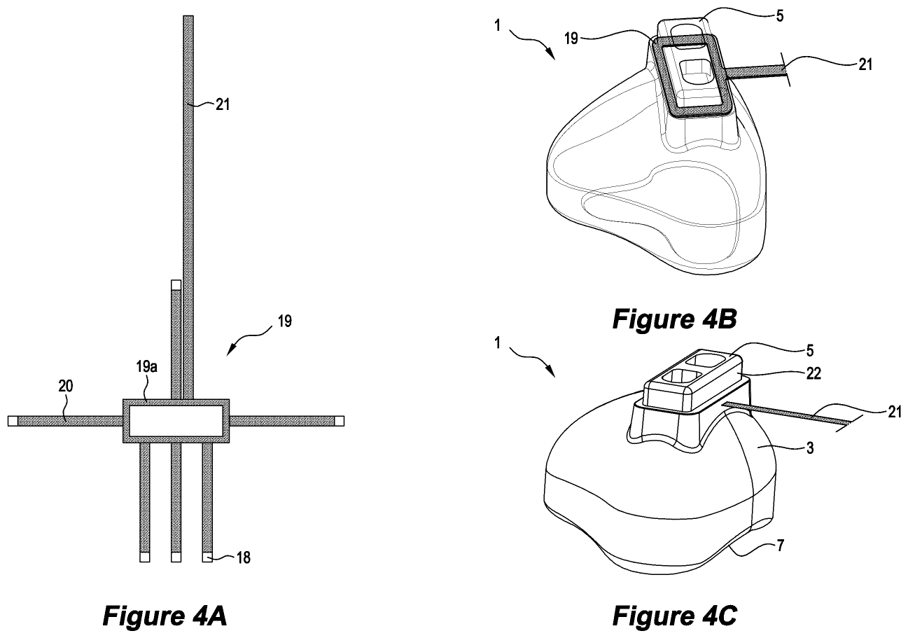

In some embodiments of the mask (1) the plurality of sensors (17) can be in direct contact with the subject's skin and not mounted internally of the mask (1). Measuring the force exerted by the mask (1) on the subject's skin (contact pressure) provides the medical practitioner with a real-time and objective measure to guide and improve the quality of mask application. This serves as an early warning system for instances in which the mask (1) is too tight or too loose, to prevent any adverse consequences (i.e. pressure injury or hypoxia from insufficient airway pressure support). In some embodiments this functionality is a passive system, and in some embodiments an active system that self-adjusts in response to the sensor data taken from the mask (1) described herein in relation to FIGS. 4A and 4B.

In some embodiments, the plurality of sensors (17) is embedded in the cushion (7) or cap (3) of the mask (1). It is alternatively contemplated, that a silicon (or similar medical grade material) sleeve or conformal layer could support the plurality of sensors (17), thereby allowing retrofitting of existing CPAP devices in hospitals and medical facilities.

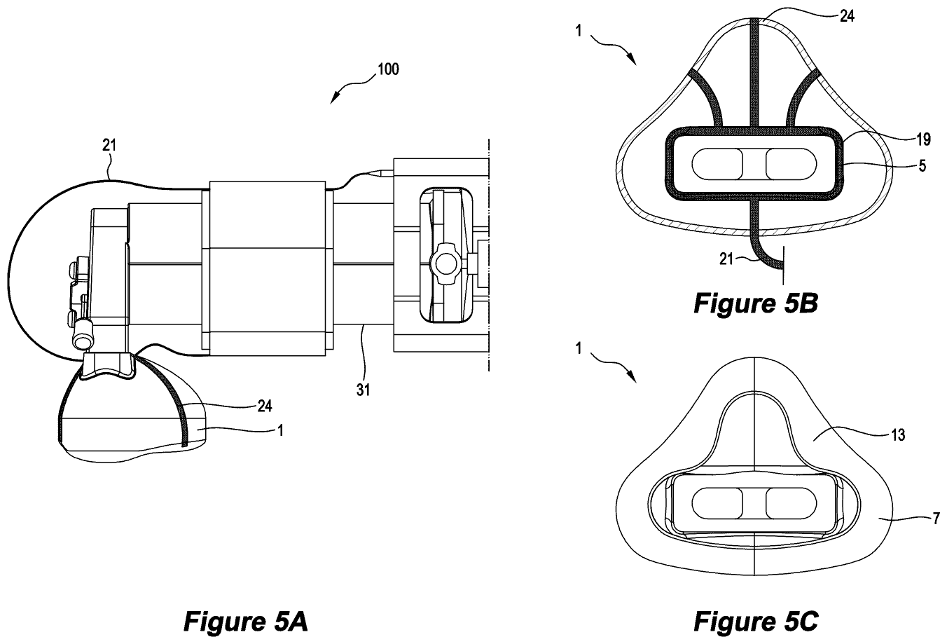

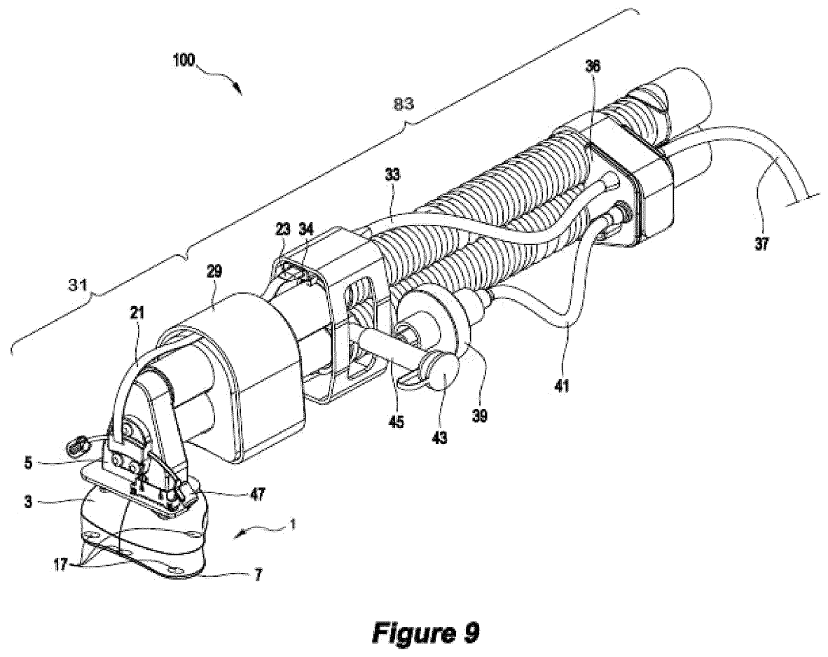

The lead (21) terminates with a connector (23) for engaging with a data port (34) to continue transmission of the sensor data. The sensor data, whether force readings, pressure readings, resistance readings etc. are directed to a computer or processor (35) for analysis and/or to a monitor (57) for viewing and/or to an alarm (61) where fluctuations of contact pressure outside a predetermined contact pressure range will trigger an alarm (see FIG. 9). Illustrated in FIG. 5, the data port (34) can be integrated with additional medical devices for use with the mask (1).

The data from each sensor (18) is sent to the pre-processor (36) for signal filtering, and then to the processor (35) housed within a monitor (57) for further processing and interpretation by the algorithm in order to display the measured contact pressures in a clinically meaningful manner: this may be quantitatively or qualitatively. The alarm (61) will be triggered when the mask (1, 101) is too tight or too loose (i.e. contact pressure out of range), or if there is a significant loss in delivered airway pressure as measured from the airway pressure sensor (55).

A default threshold range for contact pressure can be varied based on the subject's age and size. This can be determined on the gestation at birth and post-natal age, and the medical practitioner can further adjust the limits via a user interface (59) of the monitor (57).

The alarm (61) can be any one of, or combinations of, visual, audio and haptic signals. Visual alarms cannot be suppressed or hidden; however, audible alarms can be muted (for a limited period, and when the coloured LEDs are illuminated) via the mute function (69) on the monitor (57). Additionally, the audio alarm can be adjusted in volume or relayed to one or more portable electronic devices.

The alarm (61) can be integrated with other hospital systems like alarms from bedside monitors or nurse pendant monitoring systems. The data output cable (37) can also be configured to send alarms to other hospital systems, or sent text messages and alerts to enabled personal devices.

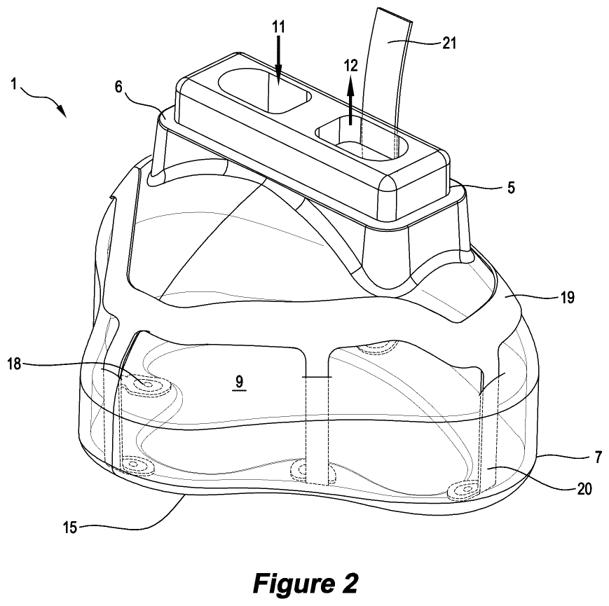

FIG. 2 is an enlarged view of the mask (1) from FIG. 1, and shows the individual sensors (18) located around the lip (15) and connected to the electrical tendrils (20). A distal end of each tendril (20) engages the sensor (18), the tendril (20) extending from the harness (19) and turning through 90 degrees to sit flush on the lip (15) inside of the chamber (9) to take readings of the contact pressure from the sealing surface (13). The electrodes (not shown) of each sensor (18) will provide a changing signal in response to changes in contact pressure e.g. a change in electrical resistance, which is then detected via the harness (19) for interpretation by the processor (35).

While FIG. 2 illustrates the harness (19) mounted on the exterior of the cap (3) and the tendrils (20) passing through the cap (3) to mount internally on the lip (15), it is contemplated that the harness (19) can be moulded into the mask (1) such that the harness becomes embedded within the material of the mask (1) and is fully encased and insulated therein.

The mask (1) can be made from soft plastics, preferably silicon or similar medical grade material. Alternatively, the cushion (7) can be made from a soft plastic or rubber to allow for deformation of the sealing surface (13), while the cap (3) and frame (5) are made from harder or rigid plastic materials to hold their shape and resist deformation of form.

In preferred embodiments, at least one of the cap, frame and cushion are made from a transparent material to allow visual monitoring of the nares (and mouth) of the subject without removing the mask (1). It is further contemplated that portions of the mask (1) for example the cap (3) or cushion (7) can be manufactured as separate components to allow for a mix-and-match assembly. For example, the cushion (7) and cap (3) can be manufactured in a plurality of different sizes to better accommodate different sizes of subject, from 20 weeks old to adult. However, fewer variants of frame (5) are required, as such, different frames (5) can be engaged with caps (3) and cushions (7) to provide a bespoke mask (1) tailored to the subject's needs. The individual components can be colour coded, or faintly colour-moulded to visually indicate different sizes.

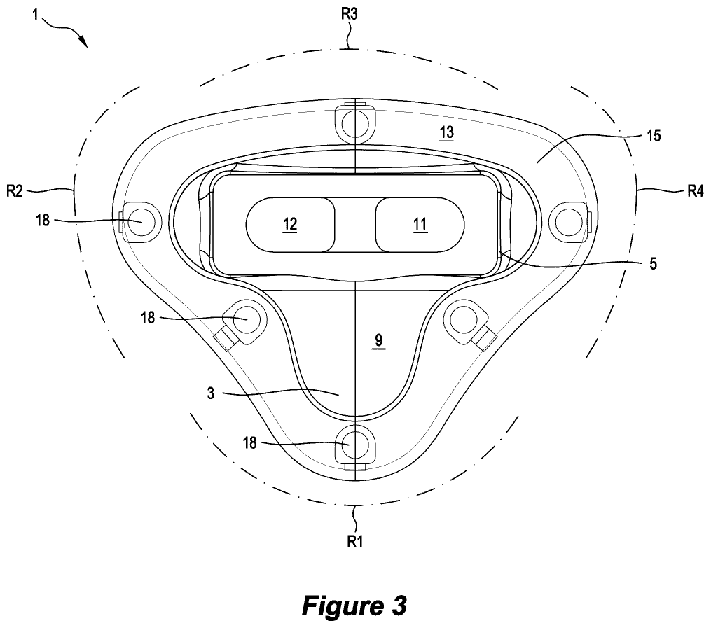

FIG. 3 is a bottom view of the mask (1) illustrating six sensors (18) in an array around the lip (15). It is contemplated that more sensors (18) can be added to the array to: provide additional data; monitor contact pressure applied to the subject in more detail; fully surround a larger mask or longer lip. Alternatively, fewer sensors (18) can be mounted in the array to provide more cost-efficient variants of the mask (1).

The array of sensors (17) are allocated to regions of the mask (1, 101) designated R1-R4, for example R1 is over the bridge of the subject's nose. Where the sensor (18) or sensors (17) designated to region 1 (R1) indicate a change in contact pressure, the medical practitioner is instantly guided to seek adjustments to the mask (1) in this region. Depending on the dimensions of the mask (1) there can be more than four regions around the lip (15) of the mask (1). Alternatively, to the embodiment shown in FIG. 3, there can be a plurality of sensors (17) disposed in each of the regions to provide more precise diagnostics on where a drop in airway pressure is occurring and which contact pressure sensors are fluctuating outside of the predetermined acceptable contact pressure range.

In FIG. 3, the lip (15) forms a perimeter around the nares of the subject forming a trilobal recess, defining a central recess for sitting over the bridge of the nose, and two opposing recesses that are configured to surround the nares of the subject. The sealing surface (13) is free from recesses and protrusions to provide a planar and low friction user interface. The sealing surface (13) will be mounted on the skin of the subject's face and form a seal with the subject's airway. The contact pressure applied to the sealing surface (13) holds the mask (1) against the subject and forms a seal therewith. This contact pressure can be manually applied by a paramedic or medical practitioner holding the mask (1) in place, or can be applied by a headband or strap that tensions the mask (1) against the subject's face. Increasing tension in the strap increases the contact pressure (i.e. force) applied to the subject via the sealing surface (13).

Where the contact pressure applied is insufficient to maintain the seal, the airway pressure and hence positive pressure air delivery to the subject can be compromised. This can lead to insufficient respiratory support for the needs of the subject. Additionally, where the contact pressure applied is excessive to that required to maintain the seal, the airway pressure is not compromised, but the sensitive facial skin around the mouth and nares of the subject can develop pressure injury, which may lead to scarring.

In some embodiments, the plurality of sensors (17) is coupled to a series of coloured LEDs providing an “in-crib” display. The LEDs can be embedded in the mask (1) or in a silicon sleeve for retrofitting over existing equipment. The “in-crib” display can be activated by a touch control on the device (100) or from the monitor (57) and can be set to automatically turn-off after a set time period.

The series of LEDs can be positioned within the chamber (9) in proximity to each sensor (18). The LEDs are configured to illuminate in response to the contact pressure applied to the subject from an adjacent sensor (18). The sensor data is transmitted to the processor (35) to assess or calculate the contact pressure applied to the subject at each sensor location and to categorise the sensor data as to whether the contact pressure exerted on the subject is within an acceptable contact pressure range.

The LEDs can be different colours to provide a visual indication of the contact pressure and trigger different alert conditions (e.g. red for too high a contact pressure, green for just right, and blue for too little contact pressure). The LEDs can be configured to only illuminate when a medical practitioner is fitting the mask (1) to the subject, providing a real-time feedback to help the medical practitioner apply the mask appropriately (as opposed to the current practice of estimating the pressure applied to the subject and continuously, manually, monitoring for any changes).

For example, when the sensor contact pressure is within the predetermined pressure range, the LEDs can emit a “green” light, visually indicating that the contact pressure on the sealing surface adjacent the LED is within the acceptable contact pressure range for the subject. When the contact pressure increases and surpasses the predetermined range, the LEDs are triggered to emit a “red” light to visually alert that the contact pressure is above the accepted range for the subject in the area adjacent the sensor (18). When the contact pressure decreases and falls short of the predetermined range, the LEDs are triggered to emit a “blue” light to visually alert that the contact pressure is below the accepted range for the subject in the area adjacent the sensor (18).

Once the mask (1) is fitted to the subject, a predetermined contact pressure range can be set for the subject. This is determined by the age and physical condition of the subject. As the plurality of sensors (17) continues to take readings, the sensor data is fed from the plurality of sensors (17) via the harness (19) and connector (23) to the processor (35). Output data from the processor is then sent to a portable device or monitor (57). In some embodiments, the processor (35) can be integrated into the monitor (57). The sensor data can be gathered continuously, or at a series of predetermined time intervals. The medical practitioner can select how much sensor data is to be gathered in selecting an operational mode of the device, from the monitor (57). Where a portable electronic device is used in place, or in combination, with the monitor, the frequency at which sensor data is gathered can be adjusted from the portable electronic device.

The processor (35) on receiving the sensor data from the lead (21) via the connector (23), calculates or assesses the contact pressure applied to the subject and then compares the contact pressure against the predetermined contact pressure range and emits output data for each sensor (18) categorised into one of three pressure states: above the predetermined contact pressure range; below the predetermined contact pressure range; and within the predetermined contact pressure reading range. In response to the pressure state afforded the sensor (18) in the output data a response is triggered.

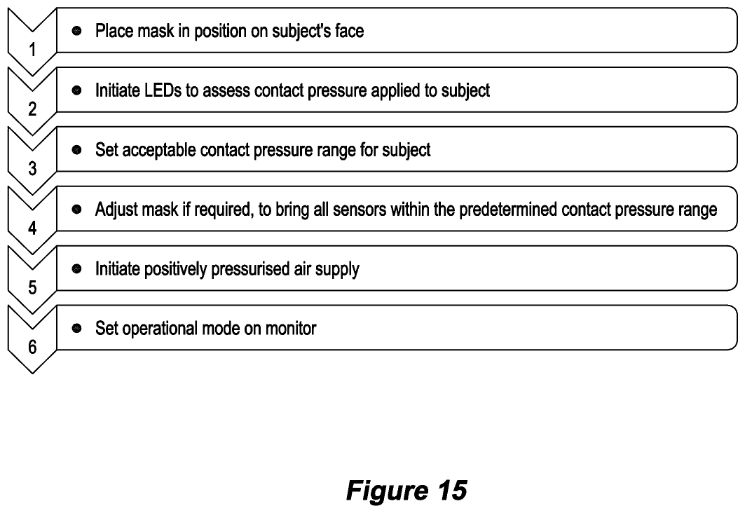

The response can be passive, taking no action where the contact pressure reading is within range, and sending an alert to the monitor or portable device notifying that the calculated contact pressure is outside of the range. The monitor (57) can be configured to display actual contact pressure values from the sensor data (or calculated contact pressures) and/or provide a qualitative display (58) visually indicating the categorisation and location of each sensor (18), see FIG. 15.

The response can be indicative, activating the LEDs to provide a visual indication of the categorisation of each sensor (18). For example, green LEDs are illuminated adjacent sensors providing a contact pressure within range, and blue and red LEDs are illuminated adjacent sensors with a contact pressure that is reduced or in excess of the predetermined contact pressure range, respectively.

The medical practitioner can select an active, indicative, or passive mode from the monitor (57) after setting-up the mask (1, 101) on the subject. The LEDs can be temporarily activated during set-up to provide the medical practitioner an instantaneous visual indication that the mask (1, 101) is correctly fitted to the subject, i.e. that the mask (1, 101) is fitted to provide optimum air flow to the subject's airways without exerting undue contact pressure on the subject.

FIG. 4A is a plan view of a six sensor harness (19), prior to its attachment to a mask (1). A central ring (19a) of the harness (19) is configured to seat onto the frame (5) of the mask (1). Extending from the central ring (19a) are the electrical tendrils (20) with a sensor (18) located at a distal end of each tendril (20). The lead (21) also extends from the central ring (19a) transmitting data from each of the sensors (18) to the connector (23) for data collection.

FIG. 4B illustrates a perspective view of the mask (in shadow outline) revealing the six sensor harness (19) mounted to the frame (5) of the mask (1), here the tendrils (20) have been omitted for clarity. FIG. 4C illustrates an opening (22) for the lead (21) of the harness (19) to exit the frame (5) of the mask (1). The harness (19) in this form can be retrofitted to existing masks. It is further contemplated that the harness (19) can comprise as few as two or three tendrils (20) with corresponding sensors (18). Alternatively, the harness (19) can comprise more than six tendrils (20) to allow 7, 8, 9, 10 or more sensors (18) to be arranged around the cushion (7) of the mask (1).

FIG. 5A is a side view of a mask (1) according to another embodiment of the disclosure, engaged with an armature delivering air under positive pressure to the mask. Shown more clearly in FIG. 5B, the cap (3) of the mask (1) is overlaid with stretch sensors (24) connecting to a harness (19) which locates and seats the stretch sensors (24) to the frame (5) of the mask (1). The harness (19) also connects a lead (21) to the stretch sensors (24) for collecting and transmitting data from the sensors (24) to the connector (23) for data collection. The stretch sensors (24) can be extended across the cap (3) and across the cushion (7) although not shown in FIG. 5C. These sensors (24) measure movement and stretch across the cap (3) of the mask (1) to indicate tension in the mask manipulation.

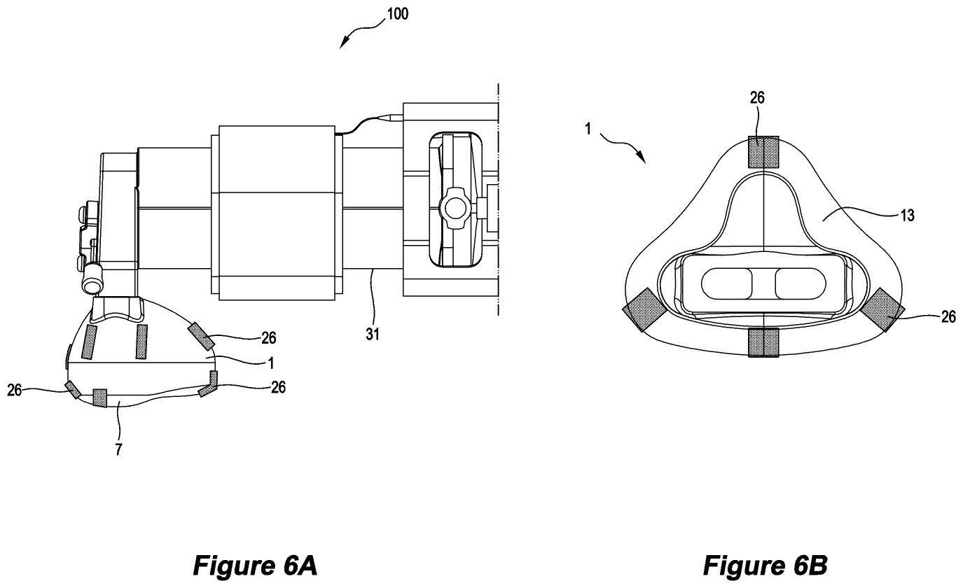

FIG. 6A illustrates a side view of a mask according to a further embodiment of the disclosure, engaged with the armature, the mask (1) providing a plurality of flex sensors (26) disposed around the cushion (7) and the cap (3). FIG. 6B is a bottom view of the mask (1) illustrating the plurality of flex sensors (26), disposed around the cushion (7). The flex sensors (26) can use any one of capacitive, resistive, piezo-resistive, and inductive sensing for assessing the flex in the cap (3) and cushion (7) of the mask (1). In some embodiments the flex sensors (26) can be confined to one or other of the cap (3) and the cushion (7) or both, as shown in FIGS. 6A and 6B. The flex sensors (26) are configured to measure deflection and deformation of the mask (1).

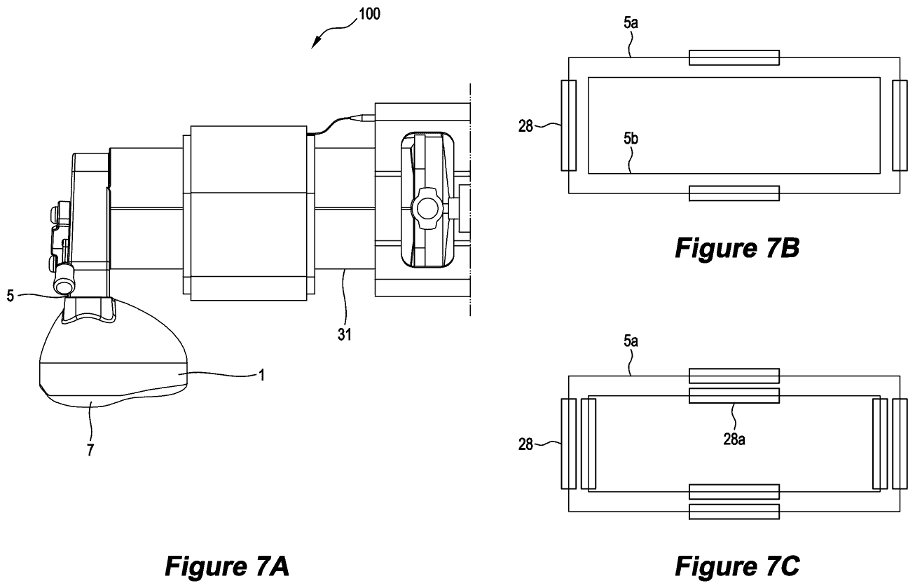

FIG. 7A is a side view of a mask according to a further embodiment of the disclosure, engaged with the armature, the mask (1) providing strain gauges (28) around the frame (5) of the mask (1). FIG. 7B is a schematic view of a cross-section through the frame (7) of the mask of FIG. 7A, indicating the location of a plurality of strain gauges (28) measuring strain on an external surface (5a) of the frame (5). In contrast, FIG. 7C is a schematic view of a cross-section through the frame (5) of the mask of FIG. 7A, indicating a plurality of strain gauges (28) measuring strain on the external surface (5a) of the frame (5) and strain gauges (28a) measuring strain on an internal surface (5b) of the frame (5) of the mask (1).

The strain gauges (28, 28a) can be integrated into the rigid cross-section of the frame (5) to measure force applied through the frame of the mask directly and thereby provide a direct indication of contact pressure applied to the subject's face. The strain gauges (28, 28a) can be integrated into the frame (5) in a 90 degree offset rosette formation, or can be mounted on opposing faces (internally and externally of the frame) to form half or full bridges configured to measure stain in an axis perpendicular to the opening of the frame (5) of the mask (1).

In relation to the stretch sensors (24), flex sensors (26) and strain gauges (28, 28a), it is contemplated that combination of some or all of these sensors can be arranged around the mask, with or without the harness (19) of sensors (18) depending on the degree of monitoring required of the mask (1).

### Active Mask

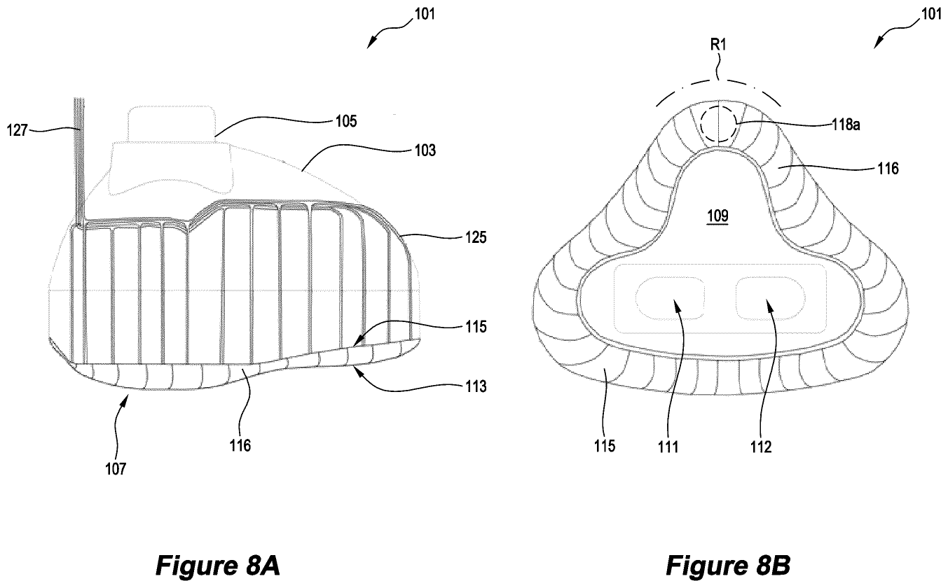

An alternative mask (101) is illustrated in FIGS. 8A and 8B. The mask (101) comprising a cap (103), a frame (105), and a cushion (107) forming a chamber (109) therein. Air inlet (111) and air outlet (112) are located in the frame (105) as described above in relation to mask (1) for connecting the mask (101) with a positively pressurised air source.

In contrast to mask (1), the cushion (107) of mask (101) is comprised of a plurality of individually controllable fluid compartments (116). The fluid compartments (116) extend across the cushion (107) of the mask (101) in contact with the subject's face. The fluid used to fill the compartments (116) can be a gas or a liquid, for example air. A plurality of sensors (117) is supported on the lip (115) of the cushion (107) internally of the chamber (109). A first sensor (118a) of the plurality of sensors (117) is illustrated in FIG. 8B disposed internally of the chamber (109) in proximity to a portion of the cushion (107) that sits atop a bridge of a nose of the subject and monitors the contact pressure applied to the subject in region #1 (R1). Region #1 is shown in dot-dashed line in FIG. 8B.

In some embodiments of mask (101) where the compartments (116) contain a fluid within a fixed space, an internal fluid pressure can be measured within each compartment (116). By determining the fluid pressure within each compartment (116) it is possible to indirectly measure the contact pressure applied to the subject's face. The fluid pressure may be measured by a pressure transducer that is not directly in the mask itself. As such, the plurality of sensors (117) in the cushion of the mask can be complemented or substituted for pressure transducers which measure the pressure of the fluid and convert the measured pressure into an electrical signal for transmission to the processor (35)

In one embodiment a discrete canister of pressurised air can be fluidly connected to each of the compartments (116) to provide a continuous air supply. The air supply to the compartments (116) is controlled by the processor (35) in response to the contact pressure measured from each sensor (18) forcing air into and out of the compartments (116) via the capillaries (125). The capillaries (125) can be opened and closed by a series of valves (not illustrated). The compartments (116) are thus automatically filled and emptied in response to the contact pressure applied to the subject's face.

As described above, the mask (101) provides an active mode that can be selected by the medical practitioner from the monitor (57). In the active mode, the mask (101) is set to continually adjust the contact pressure at each of the sensor locations in response to the sensor data. The alert system (61) and monitor display (57) can be used contemporaneously to provide visual indications and warnings where the contact pressure remains outside of the predetermined contact pressure range for a set duration.

Further to the active, indicative and passive modes, an intermittent mode can also be selected from the monitor (57) to adjust the fluid in the compartments (116) at set time intervals. In the intermittent mode, temporary relief is provided at set intervals to relieve the contact pressure applied to the subject's face for a short period as a preventative measure. For example, every ten minutes fluid in the compartments (116) is drawn out to reduce the contact pressure applied to the subject for a 1 minute duration, before re-filling the compartments (116) to bring the contact pressure back within the predetermined range. This intermittent mode can be selected after the medical practitioner has turned off the LEDs and completed the initial set-up (fitting) of the mask (1, 101).

The fluid compartments (16) extend from a lip (115) of the cushion to form a sealing surface (113) that defines a user interface with the subject. A plurality of sensors (17) is support on the lip (115) on an interior of the mask (101) to repeatedly measure the pressure (force) applied to the subject by the sealing surface (113).

Each fluid compartment (116) has a controllable volume as fluid is added and removed from any given compartment (116) by individual capillary tubes (125) that fluidly communicate with the compartments (116). The individual capillary tubes (125) are combined together into a bundle (127) that fluidly communicates with a fluid source and pump to thereby pump top-up fluid into or drawn fluid out of the compartments (116) on demand.

The compartments (116) are disposed around the periphery of the cap (103) and can be grouped into regions (see FIG. 8B where region #1 is indicated by a dot-dashed line). Each region is then cross-related to an adjacent sensor (118). For example a first sensor (118a) of the plurality of sensors (117) is located in region #1, which is cross-related with two compartments (116) in direct contact with the sensor (118a) and three compartments (116) to either side of the sensor (118a). When sensor (118a) indicates a contact pressure in excess of the predetermined contact pressure range, the pressure on the sealing surface in region #1 is too high and may cause damage to the subject. The pump is subsequently activated to drain fluid from the compartments (116) in region #1 to reduce the contact pressure applied to the subject by the sealing surface and thereby bring the contact pressure of region #1 back within the predetermined range.

Alternatively, when contact pressure at sensor (118a) falls short of the predetermined contact pressure range, the contact pressure on the sealing surface in region #1 is too low and may compromise oxygen delivery to the subject leading to trauma or asphyxia. The pump is activated to add fluid to the compartments (116) in region #1 to thereby increase the contact pressure applied to the subject by the sealing surface and bring the contact pressure in region #1 back within the predetermined range.

The contact pressure at each sensor (18) is repeatedly or continually taken and relayed to the processor (35) (via the pre-processor 36 is some cases) to assess whether the contact pressure in any given region is to be categorised as above, below or within the predetermined contact pressure range. These pressure states are then relayed in the data output to the monitor (57) in the qualitative or quantitative display (58) of the sensor array.

Effectively, the sealing surface (113) of the mask (101) can be adjusted when fitting the mask (101) to the subject to accommodate and mimic the contours of the subject's face. This provides a bespoke fit to a standard sized mask. Once fitted to the subject, the predetermined contact pressure range for the sensors (117) can be manually set or recalculated for the subject. As the sensors (117) continue to monitor contact pressure, the sensor data is fed from the sensors (117) via the harness (119) and connector (23) to the processor (35).

The processor (35) monitors or calculates the contact pressure at each sensor and compares each contact pressure against the predetermined range and determines the categorisation of the output data for each sensor into one of three pressure states: above the predetermined contact pressure range; below the predetermined contact pressure range; and within the predetermined contact pressure range. In response to the pressure states afforded the sensor (18) a response is triggered.

Where the monitor (57) is set to passive mode, the response can be passive, taking no action where the contact pressure is within range, and sending an alert or notification to the monitor (57) or portable device where the contact pressure is outside of the range. By portable device, it is understood that a pager, smart phone, table, pc or similar electronic device can be configured to receive and monitor alerts within a hospital or alternative working environment.

Where the monitor (57) is set to active mode, the response can be reactive, taking no action where the pressure reading is within range, and initiating the pump to add fluid to the compartments (116) of the cushion (107) adjacent sensors in response to contact pressure dropping below the predetermined range, and remove fluid from the sacks (116) of the cushion (107) adjacent sensors in response to contact pressure exceeding the predetermined range.

The above responses can be used in combination, where a medical professional inputs the predetermined contact pressure range using the monitor (57) or portable device, and then sets both a passive and reactive mode. The sensors (117) constantly or continually provide sensor data transmitted to the processor (35) which will augment or decrease the volume of the compartments (116) in response to the pressure state of each sensor (18) as determined in the output data. When in the active mode, there should be no reason to send a passive alert, as the system will self-regulate. However, in the event that a single sensor (118) remains outside of the predetermined reading range for a set time period (e.g. 30 seconds or more) an alert is sent to the monitor (57) alerting that the contact pressure has not been brought back into the predetermined range. This provides a failsafe mode for the mask (101).

The alert can be a visual indication, such as flashing lights or flashing images on the monitor. The alert can be an audible indicator, such as an alert or message tone, or a klaxon or ringing tone. The alert can be a haptic response on the monitor or portable device. The alert can be a combination of any one of visual, audible and haptic feedback signals. Audible alarms can be provided with a “mute” function (or button), to allow the audible alarm to be temporarily silenced while corrective action is taken.

It is contemplated that the cushion (107) can be produced as a separate component to the frame and cap of the mask (101) allowing existing masks to be retrofitted with the required fluid compartments (116). The cushion (107) is formed from a silicon sleeve including the plurality of compartments (116) and is configured to sit snuggly around a perimeter of an existing mask, meanwhile the capillaries (125) are attached or integrated into the silicon sleeve with the bundle (127) exposed for connection to a fluid source.

### CPAP Device

With reference to FIGS. 9-11, there is illustrated a continuous positive airway pressure (CPAP) device (100), comprising a mask (1) supported on an armature (31).

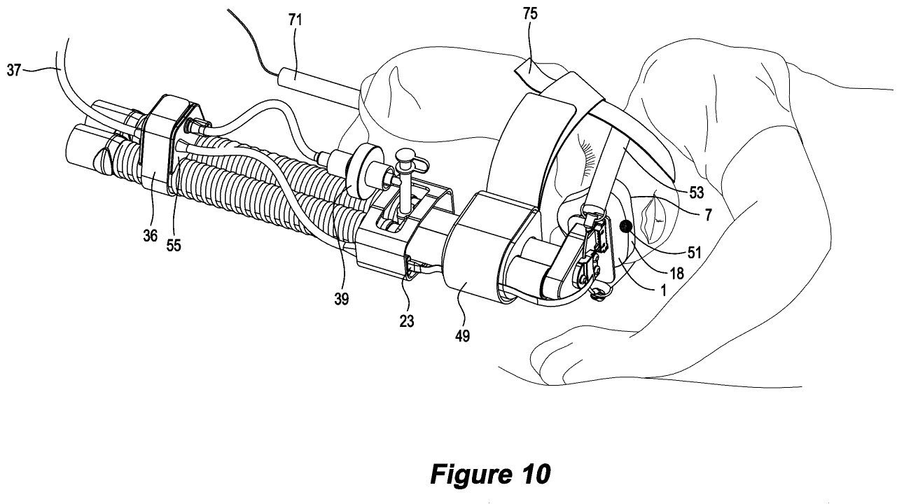

The mask (1) further comprises a tether (47) for supporting the mask (1) in an operative location relative to the subject (illustrated in FIG. 10). The mask (1) is mounted at a distal end of the armature (31) which primarily comprises an expiratory tube (81) and an inspiratory tube (79) delivering oxygenated air and removing expired air from the chamber (9) of the mask (1).

The mask (1) is engaged with the armature (31) to allow communication of positively pressurised air from the inspiratory tube (79) to the air inlet (11) and into the chamber (9). At least one constraint surrounds the armature (31) illustrated in FIG. 9 as a strap (29) detachably mounted to constrain the lead (21) to the armature and eliminate if not minimise straggling cables and leads that could snag or become entangled with the subject. The lead (21) is guided by the strap (29) and received in a data port (34) integrated with the armature (31). The data port (34) relays the sensor data from the sensors (17) via a data cable (33), to the pre-processor (36) or directly to the processor (35). The processor (35) can output to an integrated display or to the monitor (57) via an output cable (37).

Integrated into the CPAP circuit (83) is a T-piece (45) that supports at least one of an access port (43), a filter (39), and airway pressure tubing (41).

The T-piece (45) is fluidly engaged with airway pressure tubing (41) via a filter (31). The airway pressure tubing (41) is channelled to an airway pressure sensor (55) which records the airway pressure i.e., the pressure within the inspiratory limb, representing the pressure within the mask chamber (9) applied to the airway of the subject, to ensure the airway pressure is within range. The filter (31) prevents impurities and other foreign matter being drawn into the airway pressure sensor (55).

Where the airway pressure sensor (55) records a drop in airway pressure, the subject will experience a drop in the pressurisation of the air being delivered to the mask (1). This is an indication that the mask (1) is no longer sealed to the subject correctly, and is likely to be accompanied by corresponding losses in contact pressure from the array of sensors (17) or from one or more regions of the mask (1) where remedial action is required to restore the desired airway pressure. This is the most frequent reason for action on the part of clinicians supervising CPAP support in a neonatal intensive care unit.

When the airway pressure drops, the required response will depend on the quantum of the drop in airway pressure. In a passive form of device (100) a drop in circuit pressure is displayed and/or alarmed on the monitor (57). The medical practitioner will then take appropriate action (e.g., if the airway pressure drops and the sensors (18) confirm that contact pressure around the mask has reduced, then the required action is to tighten-up the mask (1). If the airway pressure drops but there is not simultaneous indication of changes in the contact pressure and all sensors (18) are within the acceptable contact pressure range, then the required action will be to check for leakages within: (i) the device (100) e.g. loose connections in the airway circuit; or (ii) from the subject e.g. the subject's mouth opening).

In an active form of the device (100) for example using mask (101), the algorithms of the processor (35) will be making the determination on the required remedial action. It is not anticipated that any change will be made in the air flow rate or prescribed airway pressure. The device (100) will activate the active components to correct any drop in airway pressure resulting from the mask (1, 101) becoming loose (as detected by the contact pressure sensors (18). Where the active device (100) cannot resolve the drop in airway pressure (or where the problem persists after ‘x’ attempts or duration), then the alarm (61) and/or display (58) would be activated to drawn the attention of nearby medical practitioners to perform additional check to the device and all supply lines to the device (100).

Medical practitioners can set/input the prescribed airway pressure acceptable maximum and acceptable minimum values into the monitor (57), to thereby set a range of acceptable airway pressure for a given subject.

The drop in airway pressure can be (and often is) directly attributed to a drop/loss in contact pressure. However, a drop in airway pressure is not always a result of a drop in contact pressure. Other causes of a drop in airway pressure can include the subject's mouth opening, loosened connections along the tubing of the pressurised air delivery circuit, and a drop in air supply source. The device (100) in an active form, e.g. using mask (101) or strap (53), can only resolve issues affecting airway pressure where the cause of the drop in airway pressure is a direct result of a drop/loss in contact pressure.

The strap (29) for securing the lead (21) to the head may be removably wrapped around the armature (31) and can be easily disconnected and removed from the device (100) for replacement and/or cleaning. The strap (29) can be affixed with a metal stud, a button, Velcro, a clip or a hook to secure it to the armature (31). Similarly affixed, the access port (43), data port (34), and pre-processor (36) can all be removably mounted along the armature (31) with straps (29′, 29″). Alternatively, all of the required components can be integrally formed in a body that defines the inspiratory and expiratory tubes. The body can connect to the required leads and data cables, or can have the required cables embedded therein.

The tether (47) may be secured to the frame (5) of the mask (1) for tensioning the mask (1) against the face of the subject via an adjustable strap (53) or head gear. LEDs may be mounted in proximity to each sensor (18) to provide continuous visual feedback on the sensor reading data and whether the pressure exerted on the sealing surface is within the predetermined range.

When configured for an infant subject, the armature (31) may support the weight of the device (100) and position the mask (1) above a crib or cot at a suitable height for the subject's head to engage with the mask (1).

The plurality of leads, cables and ports are located accessibly on the top of the armature (31) within easy reach of a medical practitioner but out of accidental contact from the subject. It is contemplated that a touch sensor can be located within any one of the straps (29, 29′, 29″) to facilitate easy actuation of the device (100) when attending to the subject. Where the medical practitioner has deemed continuous monitoring unnecessary, the device (100) can be activated on demand via the touch sensor to sporadically check the contact pressure applied to the subject by the sealing surface and subsequently deactivated.

The harness (19) may extend around the cap (3) of the mask (1) relaying sensor data to the pre-processor (36) and processor (35) via the data cable (33) and output cable (37). Adjacent each sensor (18) is at least one LED for providing a visual indication of the calculated contact pressure on the subject adjacent the sensor (18). A device (101) can be comprised of the mask (101) mounted distally on the armature (31). The device (101) can be set to passively, indicatively or actively monitor the pressure applied to the subject by the cushion of the mask (101).

FIG. 10 illustrates the device (100) operatively connected with the airway of an infant subject. The mask (1) of the device (100) is held in place over the nares of the subject by an adjustable strap (53). Although not illustrated, the adjustable strap (53) can also be engaged with the mask (1) alone to define an adjustable mask assembly without the armature (31).

### Active Strap

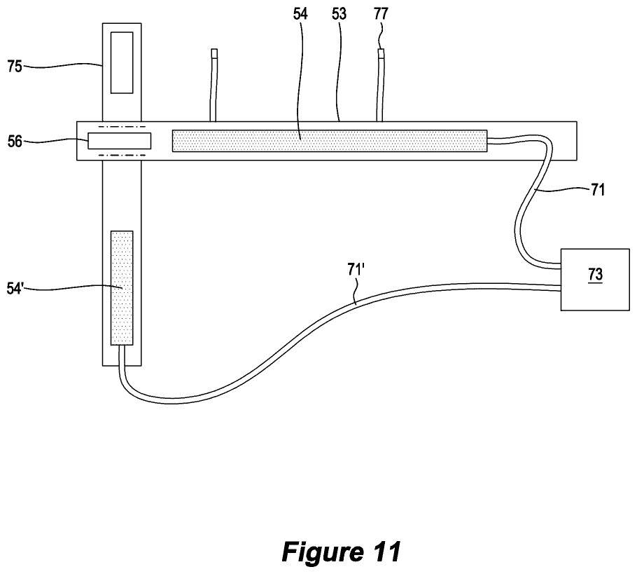

Illustrated in FIGS. 10 and 11, there is an adjustable mask assembly comprising: a mask (1) having a cap (3), a frame (5), and a cushion (7) together forming a chamber configured to be positively pressurised by a source of positive pressure air; and an adjustable strap (53) for retaining the chamber of the mask (1) adjacent to the nares of the subject, the adjustable strap (53) including a sealed cavity (54) for receiving and retaining a fluid therein, wherein introduction of the fluid into the cavity (54) increases the volume of the cavity (54) thereby tensioning the strap and increasing the regional contact pressure applied to the subject by the mask (1). In addition, the fluid can be expelled or drawn from the cavity (54) to decrease the volume of the cavity (54) and thereby decrease the regional contact pressure applied to the subject by the mask (1).

The adjustable strap (53) can also be configured for use with a mask (1) that covers the nares and mouth of the subject or alternatively for use with a full-face mask (not shown).

The adjustable strap (53) encircles the head of the subject and provides a number of tether mounts (77) for engaging with the tether (47) of the mask (1). The tether mounts (77) can terminate with snap-fit connectors for easy engagement and disengagement with the tether (47). Additionally, the tether mounts (77) can be resilient or elastic to increase the contact pressure between the mask (1) and the subject.

The adjustable strap is snuggly wrapped around the subject's head and secured in place with a connector (56). The connector (56) can be hook-and-loop (similar to Velcro™), a zip, a button, a pop-fit, a stud or similar. The tether (47) of the mask (1) is connected to the tether mounts (77) of the adjustable strap (53) and the contact pressure exerted on the subject is assessed to ensure the mask (1) is not overly tight or slack such that the positively pressurised air from the chamber (9) is efficiently delivered to the subject's respiratory system.

When the adjustable strap (53) is used in conjunction with the mask (1), the plurality of sensors (17) of the mask (1) is activated to assess the contact pressure exerted onto the subject by the mask (1). The medical practitioner can manually activate a pump (73) to pump fluid into or out of the cavity (54) to thereby adjust the tension applied by the adjustable strap (53) to the mask (1) and thereby adjust the contact pressure applied to the subject's face by the mask (1).

In a further embodiment, the sensor data is transmitted to the processor (35), which monitors or calculates the contact pressure at each sensor applied to the subject, and then compares the contact pressure at each sensor (18) against the predetermined contact pressure range and categorises each sensor into one of three pressure states; within a predetermined contact pressure range; above the predetermined contact pressure range; and below the predetermined contact pressure range.

Where the contact pressure applied to the subject is above the predetermined contact pressure range, the processor sends a signal to the pump (73) to draw fluid from the cavity (54) and thereby reduce the contact pressure applied to the subject. Alternatively, where the contact pressure applied to the subject is below the predetermined contact pressure range, the processor sends a signal to the pump (73) to pump fluid from the cavity (54) and thereby increase the contact pressure applied to the subject. The adjustable strap (53) can also be used in combination with the monitor (57) where the categorised sensors (17) are visually represented on the display (58) of the monitor (57) and the alert signal is activated when one or more of the sensors (18) continue to record contact pressures outside of the predetermined range for an extended duration, e.g. 30 seconds.

The adjustable strap (53) can include a secondary strap (75), illustrated in FIG. 10 as a chin strap. The secondary strap (75) is arranged to apply tension between the mask (1) and the subject in a different plane to that in which the adjustable strap (53) applies tension. This provides an additional degree of control in holding the mask (1) in position over the nares and controlling the contact pressure applied to the subject.

FIG. 11 is a schematic view of the adjustable strap (53) and the secondary strap (75) sewn together at right angles, such that the adjustable strap encircles the head of the subject horizontally, while the secondary strap (75) vertically encircles (at least partially) the head of the subject. Each of the two straps (53, 75) comprise at least one fluid cavity (54, 54′) connected to the pump (73) via independent fluid tubes (71, 71′). The processor (35) after categorising each sensor (18) transmits output data to activate the pump (73) sending a signal to add or remove a set volume of fluid from, or to, one or more of the cavities (54, 54′) to bring the contact pressure readings back within the predetermined contact pressure range. In some embodiments, each strap comprises a plurality of cavities, each requiring an individual fluid tube (71, 71′) to independently add or removed fluid from the cavity (54, 54′). Similar to the capillaries (25) illustrated in mask (101) a series of fluid tubes (71, 71′) can be bound into a single bundle or pipe to deliver fluid from the pump (73) to the one or more cavities (54, 54′) on demand.

Each cavity (54, 54′) can be configured to increase or decrease in volume at predetermined intervals to relieve contact pressure on the subject's skin by the straps (53, 75). Feedback signals are provided to the monitor (57) monitoring or measuring the contact pressure exerted to the subject's face by the mask (1) and/or the fluid-filled compartments (54, 54′).

Each of the adjustable straps (53, 75) comprises at least one, and in some embodiments a series of, individually fillable fluid-filled compartments or cavities (54, 54′). The fluid can be selected from the following group: air, water, warm air, warm water, cool air, and cool water.

In some embodiments, the cavities (54) are embedded within the straps. And in some embodiments, the cavities (54) are formed from bladders, externally mounted to the straps (53, 75). Using fluid filled bladders allows the system to be retro-fitted to existing headgear. The plurality of fluid tubes (71) is bound together to form a trailing bundle or pipe that can be fluidly connected to the pump (73), whether for automated or manual pumping of fluid to adjust contact pressure on the subject.

The monitor (57) allows the medical practitioner to select operative modes for the device (100). One of the available modes, is an intermittent pressure relief mode. The sensors (17) will continue to measure the contact pressure applied to the subject and categorise each sensor in relation to the predetermined range. At set intervals, the contact pressure applied to the subject is released or diminished, for a fixed duration as a preventative measure.

It is further contemplated that a servo-controlled tensioning mechanism can be incorporated into the connector (56) for further adjustments and fine tuning of the adjustable strap (53). This is a separate mechanical system which can be used if the fluid-filled cavities (54) cannot adequately adjust to the required contact pressure change.

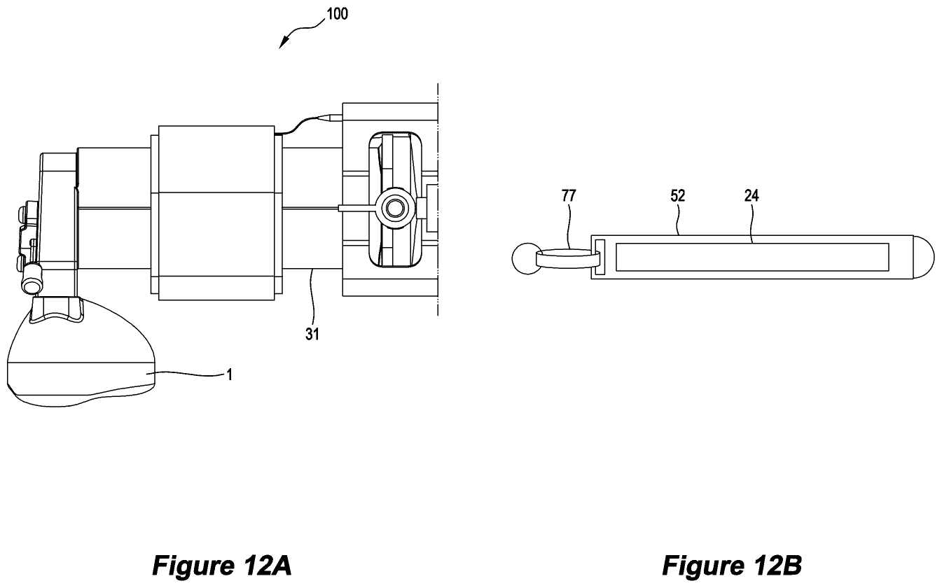

FIG. 12A is a side view of the device (100) indicating optional mounting sites on the mask (1) and armature (31) for engagement with the adjustable strap (53) or a smart strap (52) as illustrated in FIG. 12B. The smart strap (52) can comprise a stretch sensor (24) illustrated in FIG. 12B to extend along the strap (52). The sensor (24) can be disposed upon or integrated within the smart strap (52) when connected to either the armature (31) trunk or directly onto the frame (5) or cap (3) of the mask (1) to measure the tension in the strap (52) transmitted to the subject's face.

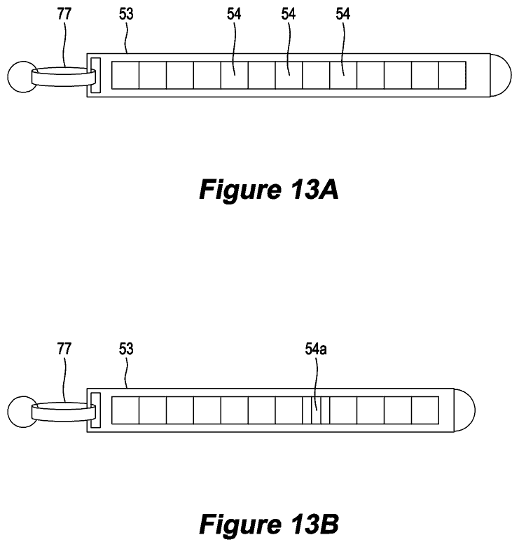

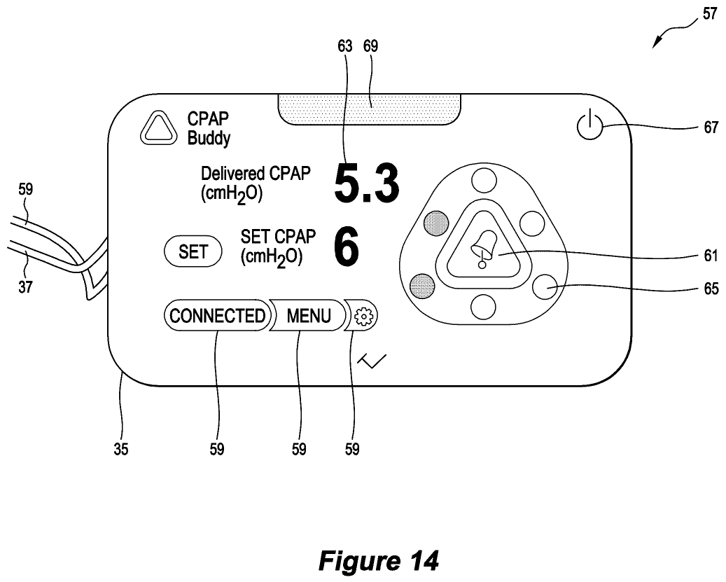

Illustrated schematically in FIG. 13A is an adjustable strap (53) according to one embodiment of the disclosure, comprising a plurality of fillable compartments (54) evenly expanded along a length of the strap (53). The volume of each of the compartments can be selectively increased or decreased (as shown in FIG. 14B having three compartments of reduced volume (54a) to vary the length of the adjustable strap (53), and thereby vary the contact pressure applied to the subject. The compartments (54) can be fluid filled using air or a liquid to provide the fluid medium for adjusting the strap (53). Using the compartments (54) to stretch/relax the strap (52) can provide a means for slight or small adjustments which can be controlled adjustments that minimise (if not eliminate) the risk of clipping or pinching the subject's skin.

In an alternative method of actuation in adjusting strap (53) the strap can contain one or more integrated muscle wires configured to vary their length/tension based on applied voltage/current. The wires would follow a similar layout to the compartments (54) illustrated in FIGS. 13A and 13B. This mechanical retraction method requires one or more in-built servomotors attached to the ends of the wires to initiate the required change in tension/length. This actuation method can provide a greater variance in the length of the strap (53) and thereby provide a greater range of adjustment. It is further contemplated that this actuation method could provide a quicker response time to the required length/tension changes within the strap (52).

FIG. 14 illustrates a monitor (57) for receiving output data from the processor (35). In some embodiments the output data can be sent directly to a portable device or smart phone and can be configured for use with an App.

The monitor provides a user interface (59) allowing a user to input data in relation to the age, size and needs of the subject. The user interface (59) can also be used to make adjustments to the predetermined contact pressure range in which the device (100) is to operate within.

The monitor (57) displays quantitative and qualitative measurements to provide continuous real-time clinical feedback to medical practitioners. The information displayed can be in absolute and/or relative terms. In some formats the measurements are colour-coded to the sensor categories; within the predetermined contact pressure range, above the predetermined contact pressure range or below the predetermined contact pressure range. The sensor array is graphically represented, as shown in FIG. 14, where red indicates excess contact pressure and blue indicates insufficient contact pressure. An alarm signal (61) is also illustrated within the graphical representation of the array which can be set to only activate when sensor readings remain outside of the predetermined range for an extended duration. The mute button (69) provides a temporary override to the alarm signal (61).

In addition to the quantitative and qualitative display (58), the monitor (57) provides: an accurate reading (63) of the measure airway pressure from the airway pressure sensor (55); a power cable (59) and an on/off button (67). The monitor (57) contains the required hardware to process the output data from the processor into clinically relatable information. In some embodiments, the processor (35) is embedded within the monitor (57).

Medical practitioners can interact with the monitor (57) via the user interface (59) for the purposes of entering clinical information which contributes to the performance for output data processing, as well as control. Monitor (57) is powered by a fixed power source, and can also contain a rechargeable battery to allow portability.

The monitor (57) can be coupled with or replaced by a third part device (mobile phone, tablet, etc.) receiving and transmitting wirelessly for secure data sharing.

The processor has a memory to store and retain data. The stored data can be displayed on command for a user-determined period and can be used as historical data for comparison against recently obtained data.

In embodiments of the mask (1, 101) or device (100) where an auto adjust mode is available, the monitor (57) provides the options for activating the desired mode of operation, i.e. setting the mechanisms necessary to control the required fine adjustments in the mask (101) and straps (53, 75).

Illustrated in the flow chart of FIG. 15 is a method of monitoring supply of positively pressurised air to a subject, the method comprising the steps of: placing a mask (1, 101) over the nares and/or mouth of the subject, the mask comprising at least one sensor (17) configured to assess and monitor contact pressure applied to the subject by the mask; transmitting data from each sensor (18) to a processor (35) for receiving or calculating the contact pressure applied to the subject by the mask (1,101); defining a predetermined contact pressure range via a monitor (57), to facilitate characterisation of the sensor into three categories: contact pressure below the predetermined range; contact pressure above the predetermined range; contact pressure within the predetermined range; initiating the supply of positively pressurised air to the mask (1, 101); and selecting an operational mode on the monitor (57) determining actions to be taken in response to the categorisation of each sensor.

In some embodiments, a plurality of sensors (17) is located spaced around the periphery of the mask (1, 101) to monitor the contact pressure applied to the subject in a plurality of locations around the mask (1, 101). In some embodiments, the sensors (17) can be embedded within the mask. Alternatively, the sensors can be mounted or embedded in a silicon sleeve that can be applied to a standard hospital mask.

Information from both the contact pressure sensors (17) and the airway pressure sensor (55) is fed to the processor (35) via the pre-processor (36) to display the effectiveness of the current mask (1, 101) application to the subject, and: (i) in the active embodiments of the disclosure, the device takes remedial action to bring the airway pressure and/or contact pressure readings back into the acceptable range for the subject; and (ii) in the passive embodiments provide an alert to a medical practitioner or clinician on what remedial action is required and where, to bring the airway pressure and/or contact pressure readings back into the acceptable range for the subject.

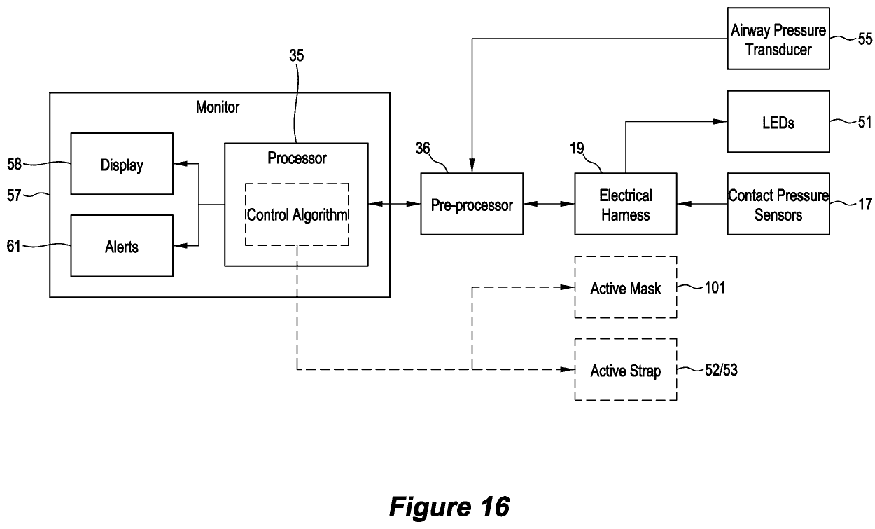

FIG. 16 is a schematic layout of the device according to one embodiment of the disclosure. The monitor (57) has the processor (35) integrated therein. The monitor (57) provides the user display (58), the user interface (59) and the alert (61). The monitor (57) receives data via the pre-processor (36). The data includes an input from the airway pressure sensor (55) which can be an airway pressure transducer converting the change in pressure detected into an electrical signal for delivering to the pre-processor (36).

The pre-processor (36) also receives data from the contact pressure sensor (18) or sensors array (17) from the mask (1, 101) and outputs data instructions to the LEDs (51) to illuminate the in-crib display.

Additional outputs from the processor (35) comprise data instruction to one or more of the active mask (101) and the active strap (52) and smart strap (53). Wherein the processor (35) on receiving data from the airway pressure sensor (55) and the contact pressure sensor/s (17/18) applies an algorithm to determine a suitable data instructions to the active mask (101) and/or active straps (52/53) to initiate remedial action in response to any one or more of: (i) a drop in airway pressure; (ii) a contact pressure reading above the predetermined maximum allowable contact pressure for the subject; and (iii) a contact pressure reading below the predetermined minimum allowable contact pressure for the subject.

Although not shown in FIG. 16, the alerts (61) can be audible and/or visual and can be generated on the display (58) of the monitor (59). The alerts (61) can also be transmitted using mobile technology, Wi-Fi, Bluetooth, BAN, PAN, NAN, LAN, WAN, CAN etc. to a mobile phone, laptop, computer or similar portable electronic device. The alerts (61) can also be configured to be sent to an existing nurse call system within the hospital or facility.

The sensors may be mounted on the mask in various ways that will be apparent to the skilled person. In some embodiments, the sensors may be embedded within the cushion.

It will be appreciated by persons skilled in the art that numerous variations and modifications may be made to the above-described embodiments, without departing from the scope of the following claims. The present embodiments are, therefore, to be considered in all respects as illustrative of the scope of protection, and not restrictively.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, a limited number of the exemplary methods and materials are described herein.

It is to be understood that, if any prior art publication is referred to herein, such reference does not constitute an admission that the publication forms a part of the common general knowledge in the art, in Australia or any other country.

In the claims which follow and in the preceding description of the disclosure, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the disclosure.

The use of the terms “a” and “an” and “the” and “at least one” and similar referents in the context of describing the disclosure (especially in the context of the following claims) are to be construed to cover both the singular and the plural, unless otherwise indicated herein or clearly contradicted by context.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

LEGEND

| #No. | Desc. |
|---|---|
| 1 | Mask |
| 2 | |
| 3 | Cap |
| 4 | |
| 5 | Frame |
| 6 | Seat |
| 7 | Cushion |
| 8 | |
| 9 | Chamber |
| 10 | |
| 11 | Air inlet aperture |
| 12 | Air outlet aperture |
| 13 | Sealing surface |
| 14a/b | |
| 15 | Lip |
| 16 | Fluid-compartments |
| 17 | Plurality of sensors |
| 18 | Contact pressure |
| | sensor |
| 19 | Harness |
| 20 | Electrical tendril |
| 21 | Conduit/Lead |
| 22 | Opening |
| 23 | Connector |
| 24 | Stretch sensor |
| 25 | Capillaries |
| 26 | Flex sensor |
| 27 | Bundle |
| 28 | Strain gauge |
| 29 | Strap |
| 30 | |
| 31 | Armature |
| 33 | Data cable |
| 34 | Data port |
| 35 | Processor |
| 36 | Pre-processor |
| 37 | Output cable |
| 38 | |
| 39 | Filter |
| 40 | |
| 41 | Airway pressure |
| | tubing |
| 42 | |
| 43 | Access port |
| 44 | |
| 45 | T-Piece |
| 46 | |
| 47 | Tether |
| 48 | |
| 49 | Touch Sensor |
| 50 | |
| 51 | LED |
| 52 | Smart strap |
| 53 | Adjustable strap |
| 54 | Fluid cavity |
| | within strap |
| 55 | Airway pressure |
| | sensor |
| 56 | Velcro tab |
| 57 | Monitor |
| 58 | Display |
| 59 | User interface |
| 60 | |
| 61 | Alarm |
| 62 | |
| 63 | Airway pressure |
| | reading |
| 65 | Sensor Display |
| 66 | |
| 67 | On/Off |
| 68 | |
| 69 | Mute alarm |
| 70 | |
| 71 | Fluid Tube |
| 72 | |
| 73 | Fluid pump |
| 74 | |
| 75 | Secondary strap |
| 76 | |
| 77 | Tether mounts |
| 78 | |
| 79 | Inspiratory tube |
| 80 | |
| 81 | Expiratory tube |
| 82 | |
| 83 | CPAP circuit |
| 84 | |
| 85 | |
| 86 | |
| 87 | |
| 88 | |
| 89 | |
| 90 | |
| F | |
| 100 | CPAP Device |

1-18. (canceled)

19. A mask for use with a positive airway pressure device, the mask comprising: a cap, a frame, and a cushion together forming a chamber configured to be positively pressurised by a source of positively pressurised air, the frame comprising an aperture for communicating the source of positively pressurised air into the chamber adjacent to the nares and/or mouth of a subject, the cushion extending around a periphery of the cap defining a lip and a sealing surface, wherein the lip supports at least one sensor and the sealing surface seals the chamber against the subject, the at least one sensor configured to assess contact pressure applied to the subject by the sealing surface of the cushion and airway pressure, wherein the mask is configured to automatically respond to suboptimal mask application or suboptimal airway pressure,
wherein the mask is fitted over the nares and/or mouth of a subject, the source of positively pressurised air is initiated to deliver positively pressurised air to the mask, the at least one sensor continuously relays data on the airway pressure and contact pressure applied to the subject by the sealing surface of the cushion,
and wherein the processor is configured to:
categorise the data on the contact pressure applied to the subject by the sealing surface of the cushion as above or below a predetermined contact pressure range;
measure airway pressure; and
categorise the measured airway pressure as satisfactory or sub-optimal.

20. The mask of claim 19, wherein the cushion supports a plurality of sensors spaced around the cushion to form a sensor array.

21. The mask of claim 20, wherein an LED is mounted adjacent to each of the plurality of sensors, the LED configured to indicate when the contact pressure as assessed by the adjacent sensor is outside of a predetermined range.

22. The mask of claim 20, wherein each of the plurality of sensors transmits sensor data to a processor, the processor determining a measure of contact pressure applied to the subject at the site of each sensor.

23. The mask of claim 22, wherein the processor creates output data categorising data from each sensor into one of three pressure states: above the predetermined contact pressure range; below the predetermined contact pressure range; and within the predetermined contact pressure range, based on the sensor data received.

24. The mask of claim 23, wherein the processor output data is directed to a monitor configured to display the pressure state for each sensor.

25. The mask of claim 23, wherein the processor output data is directed to an electronic controller configured to activate an LED mounted adjacent each of the plurality of sensors.

26. The mask of claim 23, wherein the cushion comprises a plurality of fluid-filled compartments, each compartment connected with a fluid supply to selectively augment or decrease the volume of any one or more compartments.

27. The mask of claim 26, wherein the fluid is supplied to or drawn from selected fluid-filled compartments in response to the pressure state afforded each sensor by the processor.

28. An adjustable mask assembly, comprising:
a mask according to claim 19; and
an adjustable strap for retaining a chamber of the mask adjacent to the nares and/or a mouth of a subject.

29. The adjustable mask assembly of claim 28, wherein the processor applies a mathematical algorithm to determine an automated response to aberrations in contact pressure (above or below a predetermined range) or to sub-optimal airway pressure, and the processor applies the automated adjustment of the adjustable strap or alteration of fluid volume within mask compartments to alleviate the aberrations in contact pressure or sub-optimal airway pressure.

30. The adjustable mask assembly of claim 28, wherein the adjustable strap comprising a cavity retaining a variable volume of fluid, wherein introduction of fluid into the cavity tensions the adjustable strap and thereby increases a contact pressure applied to the subject by the mask.

31. The adjustable mask assembly of claim 28, wherein a cushion of the mask comprises at least one sensor configured to assess the contact pressure between a sealing surface of the mask and the subject.

32. The adjustable mask assembly of claim 31, wherein fluid is introduced into the cavity in response to the at least one sensor determining a measure of the contact pressure between the sealing surface of the mask and the subject to be below a predetermined minimum contact pressure range.

33. The adjustable mask assembly of claim 31, wherein fluid is extracted from the cavity in response to the at least one sensor determining the contact pressure between the sealing surface of the mask and the subject is in excess of a predetermined maximum contact pressure range.

34. The adjustable mask assembly of claim 28, wherein a servo-controlled tensioning mechanism is incorporated into a connector for the adjustable strap, to provide for tensioning of the strap.

35. The adjustable mask assembly of claim 28, wherein the strap contains one or more integrated muscle wires configured to vary their length or tension based on an applied current.

36. A positive airway pressure device, comprising a mask supported on an armature the mask having a chamber for directing a positive pressure air supply to the nares and/or a mouth of the subject, and providing a plurality of sensors configured to assess contact pressure applied to the subject by the mask, the armature comprising:
a respiratory channel and an expiratory channel for supplying positively pressurised air to the chamber and evacuating expelled air therefrom; and
a processor for receiving sensor data from the plurality of sensors, and calculating the contact pressure at each sensor,
wherein the processor compares the contact pressure at each sensor against a predetermined contact pressure range to categorise the pressure state of each sensor as being: above the predetermined contact pressure range; below the predetermined contact pressure range; and within the predetermined contact pressure range.

37. The device of claim 34, wherein the pressure state of each sensor is transmitted as output data from the processor to a monitor configured to quantitatively indicate the pressure state of each sensor.

38. The device of claim 34, wherein the pressure state of each sensor is transmitted as output data from the processor to a monitor configured to qualitatively indicate the pressure state of each sensor.

39. A method of monitoring for, and automatically responding to, sub-optimal mask application or airway pressure using a positive airway pressure device comprising a mask and a source of positively pressurised air, the method comprising the steps of:
(a) fitting the mask over the nares and/or a mouth of a subject, the mask including a plurality of sensors;
(b) initiating the source of positively pressurised air to deliver positively pressurised air to the mask;
(c) continuously relaying data from the plurality of sensors to a processor to assess a contact pressure between the mask and the subject, and airway pressure;
(d) categorising data, by the processor, from each of the plurality of sensors against a predetermined contact pressure range, to assess whether the contact pressure between the mask and the user at each sensor location is: above the predetermined contact pressure range; below the predetermined contact pressure range; and within the predetermined contact pressure range;
(e) measuring airway pressure and categorising the measured pressure as satisfactory or sub-optimal by the processor.

40. The method of claim 37, further comprising:
(f) applying a mathematical algorithm to determine an automated response to aberrations in contact pressure (above or below a predetermined range) or to sub-optimal airway pressure;
(g) relaying algorithmically-determined data instructions to the processor;
(h) producing, through the algorithmically-determined data instructions to the processor, automated adjustment of adjustable straps or alteration of fluid volume within mask compartments to alleviate the disturbances in aberrations in contact pressure or sub-optimal airway pressure.
